(19) United States
(12) Patent Application Publication
(10) Pub. No.: US 2014/0220004 A1
(43) Pub. Date: Aug. 7, 2014

(54) COMPOSITIONS AND METHODS FOR TREATING PAIN

(71) Applicants: Nader Ghasemlou, Brookline, MA (US); Christian A. Von Hehn, Brookline, MA (US); Clifford J. Woolf, Newton, MA (US)

(72) Inventors: Nader Ghasemlou, Brookline, MA (US); Christian A. Von Hehn, Brookline, MA (US); Clifford J. Woolf, Newton, MA (US)

(73) Assignee: CHILDREN'S MEDICAL CENTER CORPORATION, Boston, MA (US)

(21) Appl. No.: 14/122,812

(22) PCT Filed: Jun. 1, 2012
(86) PCT No.: PCT/US2012/040397

§ 371 Date: Mar. 26, 2014

Related U.S. Application Data
(60) Provisional application No. 61/491,921, filed on Jun. 1, 2011.

(51) Int. Cl. A61K 39/395; A61K 31/713; A61K 45/06; C07K 16/18; C07K 14/47

(52) U.S. Cl. CPC A61K 39/3955; C07K 16/18; C07K 14/47; A61K 45/06; A61K 31/713
USPC 424/133.1; 424/172.1; 514/44 R; 424/158.1

(57) ABSTRACT

Provided herein are compositions containing an antibody or antigen-binding antibody fragment that specifically binds to metallothionein 2 (MT2) protein, and compositions containing an agent that binds to or neutralizes a function of MT2 protein and/or an oligonucleotide that decreases the expression of MT2 mRNA in a mammalian cell, and optionally, one or more anti-inflammatory agents and/or analgesics. Also provided are methods of decreasing pain in a mammal that include administering to the mammal an agent that binds to or neutralizes a function of MT2 protein and/or an oligonucleotide that decreases the expression of MT2 mRNA in a mammalian cell, in an amount sufficient to decrease the level of extracellular MT2 protein or neutralize a function of extracellular MT2 protein in the mammal, thereby decreasing pain in the mammal.

### BACKGROUND OF THE INVENTION

Pain is the main reason for visiting the emergency department in more than 50% of cases (Cordell et al., Am. J. Emerg. Med. 20:165-169, 2002), and is present in 30% of family practice visits (Hasselstrom et al., Eur. J. Pain 6:375-385, 2002). There are a variety of different types of pain (e.g., nociceptive pain, neuropathic pain, and inflammatory pain) and a number of different causes of pain. For example, inflammatory pain can be caused, e.g., by tissue injury, surgical wounds, arthritis, and cancer.

Pain sensitivity is also augmented during peripheral inflammation. This effect is mediated by nociceptive sensory neurons, whose cell bodies are present in the dorsal root and trigeminal ganglia, and whose peripheral terminals are specialized to respond to noxious thermal and mechanical stimuli. The nerve fibers of these neurons can become activated by various chemical mediators including endogenous cytokines and lipids, and exogenous chemical irritants. Receptors on the neuron and its peripheral fibers are able to bind these stimulatory ligands, and result in the activation of the sensory fibers, which in turn, leads to the sensation of pain.

### SUMMARY OF THE INVENTION

The invention is based, in part, on the discovery that extracellular metallothionein 2 (MT2) protein mediates pain in animal models. Based on this discovery, provided herein are compositions that contain an antibody or antigen-binding antibody fragment that specifically binds to MT2 protein, and compositions that contain an agent that binds to and/or neutralizes a function of MT2 protein and/or an oligonucleotide that decreases the expression of MT2 mRNA in a mammalian cell (e.g., a fibroblast), and optionally, one or more anti-inflammatory agents or analgesics. Also provided are methods of decreasing pain (e.g., inflammatory, neuropathic, and/or nociceptive pain) in a mammal that include administering to the mammal an antibody or antigen-binding antibody fragment that specifically binds to MT2 protein or an agent that binds to and/or neutralizes a function of MT2 protein and/or an oligonucleotide that decreases the expression of MT2 mRNA in a mammalian cell (e.g., a fibroblast), in an amount sufficient to decrease the level of extracellular MT2 protein or neutralize a function of extracellular MT2 protein in the mammal, thereby decreasing pain in the mammal Also provided are methods of identifying a candidate agent that decreases pain in a mammal that include providing a mammalian cell that expresses MT2 (e.g., a mammalian fibroblast), contacting the mammalian cell with a candidate agent, determining a test level of MT2 expression by the mammalian cell, comparing the test level of MT2 expression by the mammalian cell to a reference level of MT2 expression in a control mammalian cell that expresses MT2 untreated with the candidate agent, and selecting a candidate agent that results in a test level of MT2 expression that is lower than the reference level of MT2 expression as being useful for decreasing pain in a mammal

Provided herein are compositions (e.g., pharmaceutical compositions) that contain an antibody or antigen-binding antibody fragment that specifically binds to MT2 protein. In some embodiments, the antigen-binding antibody fragment is selected from the group consisting of: a Fab fragment, a F(ab′)₂ fragment, and a scFv fragment. In some embodiments, the antibody is a humanized antibody. In some embodiments, the antibody is an IgG or IgM antibody. In some embodiments, the antibody or antigen-binding antibody fragment has an affinity for MT2 protein that is at least 10-fold higher (e.g., at least 15-fold, 20-fold, 25-fold, 30-fold, 40-fold, 50-fold, 60-fold, 70-fold, 80-fold, 90-fold, or 100-fold higher) than its affinity for MT 1 protein.

Provided herein are compositions (e.g., pharmaceutical compositions) containing an agent that binds to and/or neutralizes a function of MT2 protein and/or an oligonucleotide that decreases the expression of MT2 mRNA in a mammalian cell, and one or more anti-inflammatory agents or analgesics. In some embodiments, the one or more anti-inflammatory agents are selected from the group of: a non-steroidal anti-inflammatory drug (NSAID), a corticosteroid, an immune selective anti-inflammatory derivative (ImSAID), and a biologic (e.g., an anti-tumor necrosis factor a antibody, an anti-interleukin-1 antibody, or an anti-nerve growth factor antibody). In some embodiments, the NSAID is a cyclooxygenase-1 (COX-1) inhibitor or a COX-II inhibitor. In some embodiments, the agent is an antibody or an antigen-binding antibody fragment that binds to or neutralizes a function of MT2 protein. In some embodiments, the antigen-binding antibody fragment is selected from the group consisting of: a Fab fragment, a F(ab′)₂ fragment, and a scFv fragment. In some embodiments, the antibody is a humanized antibody. In some embodiments, the antibody is an IgG or IgM antibody. In some embodiments, the agent is an aptamer. In some embodiments, the oligonucleotide is an inhibitory RNA (e.g., a small interfering RNA), an antisense oligonucleotide, or a ribozyme.

Also provided are methods of decreasing pain in a mammal that include administering to the mammal an agent that binds to and/or neutralizes a function of MT2 protein and/or an oligonucleotide that decreases the expression of MT2 mRNA in a mammalian cell, in an amount sufficient to decrease the level of extracellular MT2 protein and/or neutralize a function of extracellular MT2 protein in the mammal, thereby decreasing pain in the mammal In some embodiments, the pain is inflammatory pain. In some embodiments, the pain is neuropathic pain. In some embodiments, the pain is nociceptive pain. In some embodiments, the agent is an antibody or an antigen-binding antibody fragment that binds to or neutralizes MT2 protein. In some embodiments, the antigen-binding antibody fragment is selected from the group of: a Fab fragment, a F(ab′)₂ fragment, and a scFv fragment. In some embodiments, the antibody is a humanized antibody. In some embodiments, the antibody is an IgG or IgM antibody. In some embodiments, the agent is an aptamer. In some embodiments, the oligonucleotide is an inhibitory RNA (e.g., a small interfering RNA), an antisense oligonucleotide, or a ribozyme. In some embodiments, the mammal has been diagnosed as having pain. In some embodiments, the mammal is a human. In some embodiments, the administering is performed by intravenous, intraarterial, subcutaneously, intramuscular, intraarticular, epidural, intrathecal, or intraperitoneal injection. In some embodiments, the agent is administered to the mammal at least once every three months (e.g., at least once a week). In some embodiments, the mammal is administered at least one additional anti-inflammatory agent or analgesic.

Also provided are methods of identifying a candidate agent that decreases pain in a mammal that include providing a mammalian cell that expresses MT2, contacting the mammalian cell with a candidate agent, determining a test level of MT2 expression by the mammalian cell, comparing the test level of MT2 expression by the mammalian cell to a reference level of MT2 expression in a control mammalian cell that expresses MT2 untreated with the candidate agent, and selecting a candidate agent that results in a test level of MT2 expression that is lower than the reference level of MT2 expression as being useful for decreasing pain in a mammal In some embodiments, the mammalian cell that expresses MT2 is a fibroblast, a muscle cell, a keratinocyte (or other skin-resident cell), a macrophage, a T-lymphocyte, a neutrophil, or a peripheral or central nervous system glial cell. In some embodiments, the mammalian cell that expresses MT2 is in vitro. Some embodiments further include administering the selected candidate agent to an animal model of pain. In some embodiments, the mammalian cell that expresses MT2 is in a mammal In some embodiments, the test level and the reference level of MT2 expression is a level of extracellular MT2 protein. In some embodiments, the test level and the reference level of MT2 expression is a level of MT2 mRNA.

Also provided herein are methods of using an agent that binds to and/or neutralizes a function of MT2 protein and/or an oligonucleotide that decreases the expression of MT2 mRNA in a mammalian cell (e.g., a fibroblast) in the manufacture of a medicament for treating pain in a mammal

Also provided herein are agents that bind to and/or neutralize a function of a MT2 protein and/or oligonucleotides that decrease the expression of MT2 mRNA in a mammalian cell (e.g., a fibroblast) for use in treating pain in a mammal

By the term “metallothionein 2 protein” or “MT2 protein” is meant a mammalian metallothionein 2 (MT2) protein. In some embodiments, the MT2 protein is human MT2 protein (SEQ ID NO: 1).

By the term “metallothionein 2 mRNA” or “MT2 mRNA” is meant an mRNA that encodes a mammalian metallothionein 2 (MT2) protein. In some embodiments, the MT2 mRNA encodes human MT2 protein (e.g., an mRNA encoding a polypeptide of SEQ ID NO: 1, e.g., an mRNA of SEQ ID NO: 2).

By the term “function of MT2 protein” is meant the ability of MT2 protein to specifically bind to a receptor (e.g., human transient receptor potential cation channel, subfamily A, member 1) on the surface of a neuron (e.g., a nociceptive sensory neuron).

By the term “aptamer” is meant an oligonucleotide (e.g., DNA or RNA) or peptide that is capable of specifically binding to MT2 protein. In some embodiments, the aptamer is an oligonucleotide that contains one or more base, sugar, or phosphate backbone modifications. Methods for identifying an aptamer that is capable of specifically binding to a MT2 protein are known in the art.

By the term “inflammatory pain” is meant a type of pain associated with the presence of inflammation that is mediated by nociceptive sensory neurons that is sensitized by inflammatory mediators. Non-limiting causes of inflammatory pain include tissue injury, surgical wounds, arthritis, and cancer. Methods for diagnosing and treating inflammatory pain are described herein.

By the term “neuropathic pain” is meant a type of pain that is mediated by damage or disease affecting the somatosensory nervous system. Neuropathic pain may result from disorders of the peripheral nervous or central nervous system. Non-limiting subcategories of neuropathic pain include peripheral neuropathic pain, central neuropathic pain, or mixed (peripheral and central) neuropathic pain.

By the term “extracellular MT2 protein” is meant a mammalian MT2 protein that is present outside of the mammalian cell. Extracellular MT2 protein can be, e.g., identical to a form of MT2 protein found within a mammalian cell (intracellular MT2 protein).

By the term “humanized antibody” is a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (CDR) region residues of the recipient antibody are replaced by hypervariable (CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance.

In some embodiments, the humanized antibody will contain substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

By the term “stabilizing moiety” is meant a molecule that is covalently attached to an antibody or antigen-binding antibody fragment (e.g., any of the antibodies or antigen-binding antibody fragments described herein) that increases (e.g., a significant or detectable increase) the half-life or preserves the antigen-binding activity of the antibody or antigen-binding antibody fragment in vitro or in vivo. In non-limiting examples, a stabilizing moiety can be a protein, a nucleic acid, a polymer, or a carbohydrate (e.g., polysaccharide), or a combination thereof. In some embodiments, the stabilizing moiety increases the half-life or preserves the antigen-binding activity of the antibody or antigen-binding antibody fragment in vitro (e.g., in a lyophilized powder or in solution). In some embodiments, the stabilizing moiety increases the half-life or preserves the antigen-binding activity of an antibody or antigen- binding antibody fragment in vivo (e.g., in a human following administration).

By the term “specifically binds” is meant the ability of a binding molecule (e.g., an antibody, aptamer, or antigen-binding antibody fragment) to bind to a target molecule (e.g., MT2 protein) with an affinity that is greater than the affinity of the binding molecule to bind to other non-target molecules (e.g., MT1 protein). In some embodiments, an antibody or antigen-binding antibody fragment that specifically binds to MT2 protein has at least a 5-fold higher (e.g., 10-fold higher, 15-fold higher, 20-fold higher, 25-fold higher, or 30-fold higher) affinity for binding to MT2 protein compared to the antibody's or the antigen-binding antibody fragment's affinity for binding to MT1 protein.

Other definitions appear in context throughout this disclosure. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a graph showing the change in calcium flux measured in transfected human embryonic kidney (HEK) cells expressing human transient receptor potential cation channel, subfamily A, member 1 (hTRPA1) following treatment with different concentrations of rabbit metallothionein 2 A isoform containing 7 zinc atoms (MT2) (rabbit Zn7-MT2A) (Bestenbalt LLC, Estonia).

FIG. 2 is a graph showing the expression of MT2 mRNA (light bars) and MT1 (dark bars) in the hindpaw of mice 1 hour, 3 hours, 6 hours, 12 hours, 24 hours, 3 days, 7 days, and 12 days after injection with complete Freund's adjuvant (CFA).

FIG. 3 is a set of two images showing MT1/2 protein expression in the hindpaw of a control mouse not administered CFA (left panel) and MT1/2 protein expression in the hindpaw of mice 24-hours after administration of CFA (right panel).

FIG. 4A is a graph showing the expression of MT1 and MT2 mRNA in the spinal cord at different time points following CFA administration.

FIG. 4B is a graph showing the expression of MT1 and MT2 mRNA in the dorsal root ganglion at different time points following CFA administration.

FIG. 5 is a Western blot showing the expression of MT1/2 in the hindpaw of mice at different time points following CFA administration.

FIG. 6 is a graph showing the level of interleukin-1β (IL-1β) protein expression in the hindpaw of mice not administered CFA (light bar) and in the hindpaw of mice one-hour following administration of CFA (dark bar).

FIG. 7 is a Western blot showing the expression of MT1/2 secreted into the media by fibroblasts derived from the mouse hindpaw and bone marrow-derived mouse macrophages following treatment for 24-hours with different concentrations of IL-1β.

FIG. 8 is a graph showing the percentage of mustard oil and capsaicin-responsive (TRPA1+/TRPV1+), capsaicin-responsive (TRPA1−/TRPV1+), and mustard oil and capsaicin-unresponsive (TRPA1−/TRPV1−) sensory neurons showing calcium flux in response to treatment with 152 nM rabbit MT1 (rabbit Zn7-MT1) (left bar in each data set) or 152 nM rabbit MT2 (rabbit Zn7-MT2A) (right bar in each data set).

FIG. 9 is a graph showing the temporal calcium flux in all mustard oil-responsive sensory neurons in one experiment following administration of 152 nM rabbit MT2 (rabbit Zn7-MT2A), 100 μM mustard oil, and 1 μM capsaicin.

FIG. 10 is a graph showing the amount of time that mice spent licking/biting their paw (acute pain) over the first 20-minutes following injection with saline, 40 μM rabbit MT1 (rabbit Zn7-MT1), 8 μM rabbit MT2 (rabbit Zn7-MT2A), 20 μM rabbit MT2 (rabbit Zn7-MT2A), or 40 μM rabbit MT2 (rabbit Zn7-MT2A).

FIG. 11 is a graph showing the mechanical threshold needed to elicit a 50% response in mice prior to and at different time points following injection with saline, 40 μM rabbit MT1 (rabbit Zn7-MT1), 8 μM rabbit MT2 (rabbit Zn7-MT2A), 20 μM rabbit MT2 (rabbit Zn7-MT2A), or 40 μM rabbit MT2 (rabbit Zn7-MT2A).

FIG. 12 is a graph showing the mechanical threshold needed to elicit a 50% response in wild type and TRPA1 knockout littermate mice prior to and at different time points following injection with 40 μM rabbit MT2 (rabbit Zn7-MT2A).

FIG. 13 is a graph showing a whole cell recording of an HEK cell transfected with hTRPA1 using a voltage step protocol to measure the changes in current with application of rabbit MT2 (rabbit Zn7-MT2A).

FIG. 14 is a graph showing the time course of a whole cell recording of an HEK cells transfected with hTRPA1 during a wash step with 145 mM NaCl, 5 mM KCl, 2 mM CaCl₂, 1 mM MgCl₂, 10 mM glucose, 10 mM Hepes, pH 7.4; treatment with 1 μg/mL (152 nM) rabbit MT2 (rabbit Zn7-MT2A); a second wash step; 100 μM mustard oil (MO); and 10 μM ruthenium red (RR).

FIG. 15 is a graph showing the mechanical threshold needed to elicit a 50% response in wild type and MT1/2 knockout mice at different time points following injection of CFA.

FIG. 16 is a graph showing the time to response to radiant heat in wild type and MT1/2 knockout mice at different time points following heat exposure.

FIG. 17 is a graph showing the paw size in wild type and MT1/2 knockout mice prior to treatment and at different time points following CFA injection.

FIG. 18 shows fluorescence assisted cell sorting data showing the populations of myeloid immune cells, neutrophils, and inflammatory macrophages in untreated MT1/2 knockout mice (left panels), wild type mice injected with CFA (center panels), and MT1/2 knockout mice injected with CFA (right panels).

FIG. 19 is a graph showing the mechanical threshold needed to elicit a 50% response in mice at different time points following an initial injection of CFA, and subsequent injections of either 10 μl saline (squares) or 10 μl of an anti-MT1/2 antibody (130 mg/L; Dako, Inc.) (circles). The arrows indicate the time point of each subsequent injection with saline or anti-MT1/2 antibody.

FIG. 20 is a graph showing the mechanical threshold needed to elicit a 50% response in wild type (circles) and MT1/2 knockout (squares) mice in a spared injury nerve model of neuropathic pain at different time points following injury.

FIG. 21 is a graph showing the response time of wild type (circles) and MT1/2 knockout mice (squares) in a cold response (acetone) test at different time points following spared nerve injury model of neuropathic pain at different time points following injury.

### DETAILED DESCRIPTION OF THE INVENTION

The invention is based, in part, on the discovery that extracellular MT2 protein mediates pain in an animal model. Thus, provided herein are compositions (e.g., pharmaceutical compositions) that contain an antibody or antigen-binding antibody fragment that specifically binds to MT2 protein, and compositions that contain an agent that binds to MT2 protein and/or neutralizes a function of MT2 protein, and/or an oligonucleotide that decreases the expression of MT2 mRNA in a mammalian cell (e.g., a fibroblast). The compositions described herein can optionally include at least one anti-inflammatory agent or analgesic. Also provided are methods of treating pain in a mammal that include administering to a mammal an antibody or antigen-binding antibody fragment that specifically binds to MT2 protein, or an agent that binds to MT2 protein and/or neutralizes a function of MT2 protein and/or an oligonucleotide that decreases the expression of MT2 mRNA in a mammalian cell (e.g., a fibroblast). Also provided are methods of identifying a candidate agent that decreases pain in a mammal that include contacting a mammalian cell that expresses MT2 (e.g., a mammalian fibroblast) with a candidate agent and determining the level of MT2 expression in the mammalian cell. Various, non-limiting features of each aspect of the invention are described below.

### Metallothionein 2 (MT2)

MT2 is a ubiquitously expressed protein. MT2 protein has the ability to sequester up to 7 metal ions (e.g., zinc, cadmium, and copper). Non-limiting examples of MT2 proteins are endogenous MT2 proteins, e.g., an endogenous human MT2 protein (e.g., an MT2 protein containing the sequence of SEQ ID NO: 1), an endogenous dog MT2 protein (SEQ ID NO: 3), an endogenous cow MT2 protein (SEQ ID NO: 5), an endogenous rabbit MT2 protein (SEQ ID NO: 7), an endogenous mouse MT2 protein (SEQ ID NO: 9), and an endogenous monkey protein (SEQ ID NO: 11). In some embodiments, an endogenous form of MT2 protein contains a sequence that is at least 80% identical (e.g., at least 85%, 90%, 91%, 92%, 93%, 94%, 95%, 96%, 97%, 98%, 99%, or 100% identical) to SEQ ID NO: 1, 3, 5, 7, 9, or 11. A number of additional endogenous mammalian forms of MT2 protein are known in the art. The extracellular MT2 protein is the same or at least 90% identical (e.g., at least 91%, 92%, 93%, 94%, 95%, 96%, 97%, 98%, 99%, or 100% identical) to the intracellular MT2 protein.

Exemplary MT2 proteins include for example, the following proteins:

Human MT2 Protein
(SEQ ID NO: 1)
mdpncscaag dsctcagsck ckeckctsck ksccsccpvg
cakcaqgcic kgasdkcscc a
Dog MT2 protein
(SEQ ID NO: 3)
mdpncscaag gsctcagsck ckecrctsck ksccsccpvg
cakcaqgcic kgasdkcscc a
Cow MT2 protein
(SEQ ID NO: 5)
mdpncsctag esctcagsck ckdckcasck ksccsccpvg
cakcaqgcvc kgasdkcscc a
Rabbit MT2 protein
(SEQ ID NO: 7)
mdpncscptg gscscagsct ckacrcpsck ksccsccpvg
cakcaqgcvc kgasdkcscc a
Mouse MT2 protein
(SEQ ID NO: 9)
mdpncscasd gscscagack ckqckctsck ksccsccpvg
cakcsqgcic keasdkcscc a
Monkey MT2 protein
(SEQ ID NO: 11)
mdpncscvag dsctcagsck ckeckctsck ksccsccpvg
cakcaqgcic kgasdkcncc a

Non-limiting examples of MT2 mRNA that encode human, dog, cow, rabbit, mouse, and monkey MT2 are provided below. In some embodiments, the MT2 mRNA contains a sequence that is at least 80% identical (e.g., at least 85%, 90%, 91%, 92%, 93%, 94%, 95%, 96%, 97%, 98%, 99%, or 100% identical) to SEQ ID NO: 2, 4, 6, 8, 10, or 12. Additional examples of MT2 mRNA that encode other endogenous forms of mammalian MT2 are known in the art.

| Human MT2 mRNA | |
|---|---|
| (SEQ ID NO: 2) | |
| 1 | atggacccca actgctcgtg cgccgccggt gactcctgca cctgcgccgg ctcctgcaaa |
| 61 | tgcaaagagt gcaaatgcac ctcctgcaag aaaagctgct gctcctgctg ccctgtgggc |
| 121 | tgtgccaagt gtgcccaggg ctgcatctgc aaaggggcgt cggacaagtg cagctgctgc |
| 181 | gcctgatgct gggacagccc gctcccagat gtaaagaacg cgacttccac aaacctggat |
| 241 | tttttatgta caaccctgac cgtgaccgtt tgctatattc ctttttctat gaaataatgt |
| 301 | gaatgataat aaaacagctt tgtcttg |
| Dog MT2 mRNA | |
| (SEQ ID NO: 4) | |
| 1 | agcgccgccg tctctccgcc cgctgtcccg gactccagcc gccccttctc gccatggatc |
| 61 | ccaactgctc ctgcgccgcg gggggctcct gcacgtgcgc cggctcctgc aaatgcaaag |
| 121 | agtgcagatg cacctcctgc aagaagagct gctgctcctg ctgccccgtg ggctgtgcca |
| 181 | agtgtgccca gggctgcatc tgcaagggcg catcggacaa gtgcagctgc tgtgcctgat |
| 241 | gtgggggaga gcctattcct gatgtaaata gagcgacgtg tacaaaccta cagtttgtgg |
| 301 | ggggtttttt ggtgcttttt gttttgggtc caactctgac ccgtttgcta ctacattcct |
| 361 | agttattttc cctatgaaat aatacgtgaa ttataataaa agctgtcgac tttaaaaaaa |
| 421 | aaaaaaaaaa |
| Cow MT2 mRNA | |
| (SEQ ID NO: 6) | |
| 1 | gctccagcac gccccttcga caccctcgcc atcctttgct cagcagtctc cggaccccag |
| 61 | cctccagttc agctcgccat ggatcccaac tgctcctgca ccgcgggtga atcctgcacg |
| 121 | tgtgccggct cctgcaaatg caaagattgc aagtgcgcct cctgcaagaa gagctgctgc |
| 181 | tcctgctgcc ccgtgggctg tgccaagtgt gcccagggct gcgtctgcaa aggggcttcg |
| 241 | gacaagtgca gctgctgtgc ctgaaggcgg ggagagcctg ctccccggtg taaatagaac |
| 301 | aacgtgtaca aacctgcata tttttttttt aatacaacct gacccgtttg ttacatccct |
| 361 | gttttttttt tttttttttg tcctataaaa tacaagaatg ataataaaac tggttggctt |
| 421 | t |
| Rabbit MT2 mRNA | |
| (SEQ ID NO: 8) | |
| 1 | atggatccca actgctcctg ccccactggc ggctcctgca gctgtgctgg ctcctgcacc |
| 61 | tgcaaggcct gcagatgtcc ctcctgcaag aagagctgct gctcctgctg ccctgtgggc |
| 121 | tgtgccaagt gtgcccaggg ctgtgtctgc aaaggggcct cggacaagtg cagctgctgc |
| 181 | gcctga |
| Mouse MT2 mRNA | |
| (SEQ ID NO: 10) | |
| 1 | ggtcgtgcgc aggcccaggg gcgtgtgctg gccatatccc ttgagccaga aaaagggcgt |
| 61 | gtgcaggcgg cgggggcgcg tgcatggtgc cttccacccg ggcggagctt ttgcgctcga |
| 121 | cccaatactc tccgctataa aggtcgcgct ccgcgtgctt ctctccatca cgctcctaga |
| 181 | actcttcaaa ccgatctctc gtcgatcttc aaccgccgcc tccactcgcc atggacccca |
| 241 | actgctcctg tgcctccgat ggatcctgct cctgcgctgg cgcctgcaaa tgcaaacaat |
| 301 | gcaaatgtac ttcctgcaag aaaagctgct gctcctgctg ccccgtgggc tgtgcgaagt |
| 361 | gctcccaggg ctgcatctgc aaagaggctt ccgacaagtg cagctgctgt gcctgaaggg |
| 421 | gggcggaggg gtccccacat ctgtgtaaat agaccatgta gaagcctagc cttttttgta |
| 481 | caaccctgac tcgttctcca caactttttc tataaagcat gtaactgaca ataaaagccg |
| 541 | ttgacttgat taattc |
| Monkey MT2 mRNA | |
| (SEQ ID NO: 12) | |
| 1 | cccgactcca gccgcctctt caactcgcca tggatcccaa ctgctcttgc gtcgccggtg |
| 61 | actcctgcac ctgcgccggc tcctgcaagt gcaaagagtg caaatgcacc tcctgcaaga |
| 121 | aaagctgctg ctcctgctgc cctgtgggct gtgccaagtg tgcccagggc tgcatctgca |
| 181 | aaggggcgtc ggacaagtgc aactgctgcg cctgatgctg ggacagccct gctcccagat |
| 241 | gtaaataatg cgacctctac aaacctggat ttttttatgt acaaccctga tcgtttgctg |
| 301 | cattcctttt tctatgaaat aatatgaatg ataataaaac agctttgacg |

### Compositions and Kits

Provided herein are compositions (e.g., pharmaceutical compositions) that contain an antibody or antigen-binding antibody fragment that specifically binds to MT2 protein, and compositions that contain an agent that binds to and/or neutralizes a function of MT2 protein, and/or an oligonucleotide that decreases the expression of MT2 mRNA in a mammalian cell (e.g., a mammalian fibroblast). Some embodiments of these compositions further contain one or more anti-inflammatory agents and/or analgesics. Various exemplary aspects of these compositions are described below. One or more of any of the various exemplary aspects of these compositions can be used in any combination.

Agents that Bind to and/or Neutralize a Function of MT2 Protein

Antibodies and Antigen-Binding Antibody Fragments

Non-limiting examples of agents that bind to and/or neutralize a function of MT2 protein include antibodies and antigen-binding antibody fragments. In some embodiments, the antibody or antigen-binding antibody fragment that binds to and/or neutralizes a function of MT2 protein can also bind to MT1 protein (e.g., an antibody or antigen-binding antibody fragment that binds to both human MT2 protein and human MT1 protein). In some embodiments, the antibody or antigen-binding antibody fragment specifically binds to MT2 protein. Non-limiting examples of antibodies that can bind to MT2 protein are the anti-metallothionein antibody (UC1MT) available from Abcam (Cambridge, Mass.), the anti-metallothionein antibody (E9) available from Dako (Carpinteria, Calif.) and the anti-MT2 antibody available from Uscn Life Science, Inc. (Missouri City, Tex.).

Methods for determining the ability of an antibody or antigen-binding antibody fragment to bind to and/or neutralize a function of a MT2 protein may be performed using the methods described herein and methods known in the art. Non-limiting examples of such methods include competitive binding assays using antibodies known to bind to MT2 protein (e.g., the anti-metallothionein antibody (UC1MT) available from Abcam), such as enzyme-linked immunosorbent assays (ELISAs), BioCoRE®, affinity columns, immunoblotting, or protein array technology. The ability of an agent to neutralize a function of a MT2 protein can be performed by determining the ability of an agent to prevent or decrease the ability of a MT2 protein to bind to the surface of a neuron (e.g., a nociceptive sensory neuron) or to prevent or decrease the ability of a MT2 protein to stimulate calcium flux in a neuron (e.g., a nociceptive sensory neuron). In some embodiments, the binding activity of the antibody or antigen-binding antibody fragment is determined by contacting a purified MT2 protein (e.g., SEQ ID NO: 1) or a peptide fragment thereof, with the antibody or antigen-binding antibody fragment.

In some embodiments, the antibody or antigen-binding antibody fragment binds to MT2 protein (e.g., human MT2 protein) with an K_{D} equal to or less than 1×10⁻⁷ M, a K_{D} equal to or less than 1×10⁻⁸ M, a K_{D} equal to or less than 5×10⁻⁸ M, a K_{D} equal to or less than 5×10⁻⁹ M, a K_{D} equal to or less than 2×10⁻⁹ M, or a K_{D} equal to or less than 1×10⁻⁹ M under physiological conditions (e.g., in phosphate buffered saline).

An antibody can also be a single-chain antibody (e.g., as described herein). An antibody can be a whole antibody molecule (e.g., a human, humanized, or chimeric antibody) or a multimeric antibody (e.g., a bi-specific antibody). An antibody or antigen-binding antibody fragment may be a variant (including derivatives and conjugates) of an antibody or an antigen-binding antibody fragment. An antibody or an antigen-binding antibody fragment may also be a multi-specific (e.g., bi-specific) antibody or antigen-binding antibody fragment. Examples of antibodies and antigen-binding antibody fragments include, but are not limited to: single-chain Fvs (sdFvs), Fab fragments, Fab′ fragments, F(ab′)₂, disulfide-linked Fvs (sdFvs), Fvs, and fragments containing either a VL or a VH domain. A single chain Fv or scFv is a polypeptide containing at least one VL domain of an antibody linked to at least one VH domain of an antibody.

Antibodies useful in the present invention include, e.g., polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding antibody fragments thereof. The antibodies or antigen-binding antibody fragments can be of any type (e.g., IgG, IgE, IgM, IgD, IgA, and IgY), class (e.g., IgG₁, IgG₂, IgG₃, IgG₄, IgA₁, and IgA₂), or subclass. In some embodiments, the antibody or antigen-binding antibody fragment is an IgG₁ antibody or antigen-binding fragment thereof. In other embodiments, the antibody or antigen-binding antibody fragment is an IgG₄ antibody or antigen-binding fragment thereof. Immunoglobulins may have both a heavy and light chain.

An isolated fragment of a MT2 protein (e.g., a fragment of a human MT2 protein) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to a protein that is immunogenic in the species to be immunized, e.g., keyhole limpet hemocyanin, serum albumin, bovine thyroglobulin, or soybean trypsin inhibitor using a bifunctional or derivitizing agent, for example, malimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Animals can be injected with the antigenic peptide or protein more than one time (e.g., twice, three times, or four times).

An exemplary MT2 protein that may be used to generate polyclonal or monoclonal antibodies are described herein (e.g., SEQ ID NO: 1). In some embodiments, a full-length MT2 protein can be used or, alternatively, antigenic MT2 peptide fragments can be used as immunogens. The antigenic peptide of a protein comprises at least 8 (e.g., at least 10, 15, 20, or 30) amino acid residues of the amino acid sequence of a MT2 protein (e.g., at least 8 amino acid residues of SEQ ID NO: 1) and encompasses an epitope of the MT2 protein, such that an antibody raised against the peptide forms a specific immune complex with the MT2 protein.

An immunogen typically is used to prepare antibodies by immunizing a suitable mammal (e.g., human or transgenic animal expressing at least one human immunoglobulin locus). An appropriate immunogenic preparation can contain, for example, a recombinantly-expressed or a chemically-synthesized polypeptide (e.g., a human MT2 protein (e.g., SEQ ID NO: 1) or a fragment of human MT2 protein (e.g., a fragment of SEQ ID NO: 1). The preparation can further include an adjuvant, such as Freund's complete or incomplete adjuvant, or a similar immunostimulatory agent.

Polyclonal antibodies can be prepared as described above by immunizing a suitable mammal with a MT2 protein, or an antigenic peptide thereof (e.g., a fragment of MT2 protein containing at least 8 amino acids) as an immunogen. The antibody titer in the immunized mammal can be monitored over time by standard techniques, such as with an enzyme-linked immunosorbent assay (ELISA) using the immobilized MT2 protein or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the mammal and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, N.Y.). Hybridoma cells producing a monoclonal antibody are detected by screening the hybridoma culture supernatants for antibodies that bind the polypeptide or epitope of interest, e.g., using a standard ELISA assay.

As an alternative to preparing monoclonal antibody-secreting hybridomas, a monoclonal antibody directed against a polypeptide can be identified and isolated by screening a recombinant combinatorial immunoglobulin library (e.g., an antibody phage display library) with the polypeptide or a peptide fragment containing the epitope of interest. Kits for generating and screening phage display libraries are commercially available (e.g., the Pharmacia Recombinant Phage Antibody System, Catalog No. 27-9400-01; and the Stratagene SurfZAP* Phage Display Kit, Catalog No. 240612). Additionally, examples of methods and reagents particularly amenable for use in generating and screening an antibody display library can be found in, for example, U.S. Pat. No. 5,223,409; WO 92/18619; WO 91/17271; WO 92/2079; WO 92/15679; WO 93/01288; WO 92/01047; WO 92/09690; WO 90/02809; Fuchs et al., Bio/Technology 9:1370-1372, 1991; Hay et al., Hum. Antibod. Hybridomas 3:81-85, 1992; Huse et al., Science 246:1275-1281, 1989; and Griffiths et al., EMBO J. 12:725-734, 1993.

In some embodiments of any of the methods described herein, the antibodies are human antibodies, humanized antibodies, or chimeric antibodies that contain a sequence from a human antibody (e.g., a human immunoglobulin constant domain and/or human immunoglobulin variable domain framework regions). Humanized antibodies are chimeric antibodies that contain a minimal sequence derived from non-human (e.g., mouse) immunoglobulin. In some embodiments, a humanized antibody is a human antibody that has been engineered to contain at least one complementary determining region (CDR) present in a non-human antibody (e.g., a mouse, rat, rabbit, or goat antibody). In some embodiments, the humanized antibody or fragment thereof can contain all three CDRs of a heavy chain of a non-human monoclonal antibody that binds to MT2 (e.g., human MT2 protein) and all three CDRs of a light chain of a non-human monoclonal antibody that binds to MT2 (e.g., human MT2 protein). In some embodiments, the framework region residues of the human immunoglobulin are replaced by corresponding non-human (e.g., mouse) antibody residues. In some embodiments, the humanized antibodies can contain residues which are not found in the human antibody or in the non-human (e.g., mouse) antibody. Methods for making a humanized antibody from a non-human (e.g., mouse) monoclonal antibody are known in the art. Additional non-limiting examples of making a chimeric (e.g., humanized) antibody are described herein.

In some embodiments, the antibodies are chimeric antibodies that contain a light chain immunoglobulin that contains the light chain variable domain of a non-human antibody (e.g., a mouse antibody) or at least one CDR of a light chain variable domain of a non-human antibody (e.g., a mouse antibody) and the constant domain of a human immunoglobulin light chain (e.g., human κ chain constant domain). In some embodiments, the antibodies are chimeric antibodies that contain a heavy chain immunoglobulin that contains the heavy chain variable domain of a non-human (e.g., a mouse antibody) or at least one CDR of a heavy chain variable domain of a non-human (e.g., a mouse antibody) and the constant domain of a human immunoglobulin heavy chain (e.g., a human IgG heavy chain constant domain). In some embodiments, the chimeric antibodies contain a portion of a constant (Fc domain) of a human immunoglobulin.

In some embodiments, the antibodies or antigen-binding fragments thereof can be multi-specific (e.g., multimeric). For example, the antibodies can take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers within an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher- order antibody multimers. Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) (available, for example, from Pierce Biotechnology, Inc., Rockford, IL) can be used to form antibody multimers. An exemplary protocol for the formation of antibody homodimers is described in Ghetie et al. (Proc. Nall. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers (see, for example, WO 96/27011).

Bi-specific antibodies include cross-linked or heteroconjugate antibodies. For example, one of the antibodies in the heteroconjugate can be coupled to avidin and the other to biotin. Heteroconjugate antibodies can also be made using any convenient cross-linking methods. Suitable cross-linking agents are well known in the art and are disclosed in U.S. Pat. No. 4,676,980, along with a variety of cross-linking techniques.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Additional methods have been developed to facilitate the direct recovery of Fab′-SH fragments from E. coli, which can be chemically coupled to form bi-specific antibodies. Shalaby et al. (J. Exp. Med. 175:217-225, 1992) describes the production of a fully-humanized bi-specific antibody F(ab′)₂ molecule. Each Fab′ fragment was separately secreted from E. coli and subjected to direct chemical coupling in vitro to form the bi-specific antibody.

Additional techniques for making and isolating bi-specific antibody fragments directly from recombinant cell culture have also been described. For example, bi-specific antibodies have been produced using leucine zippers (Kostelny et al., J. Immunol. 148:1547- 1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers.

The diabody technology described by Hollinger et al. (Proc. Natl. Acad. Sci. U.S.A. 90:6444-6448, 1993) is an additional method for making bi-specific antibody fragments. The fragments contain a heavy chain variable domain (V_{H}) connected to a light chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another method for making bi-specific antibody fragments by the use of single-chain Fv (sFv) dimers has been described in Gruber et al. (J. Immunol. 153:5368, 1994). Alternatively, the bi-specific antibody can be a “linear” or “single-chain antibody” produced using the methods described, for example, in Zapata et al. (Protein Eng. 8:1057-1062, 1995). In some embodiments the antibodies have more than two antigen-binding sites. For example, tri-specific antibodies can be prepared as described in Tutt et al. (J. Immunol. 147:60, 1991).

Alternatively, antibodies can be made to multimerize through recombinant DNA techniques. IgM and IgA naturally form antibody multimers through the interaction with the mature J chain polypeptide. Non-IgA or non-IgM molecules, such as IgG molecules, can be engineered to contain the J chain interaction domain of IgA or IgM, thereby conferring the ability to form higher order multimers on the non-IgA or non-IgM molecules (see, for example, Chintalacharuvu et al., Clin. Immunol. 101:21-31, 2001, and Frigerio et al., Plant Physiol. 123:1483-1494, 2000). IgA dimers are naturally secreted into the lumen of mucosalined organs. This secretion is mediated through the interaction of the J chain with the polymeric IgA receptor (pIgR) on epithelial cells. If secretion of an IgA form of an antibody (or of an antibody engineered to contain a J chain interaction domain) is not desired, it can be greatly reduced by expressing the antibody molecule in association with a mutant J chain that does not interact well with pIgR (Johansen et al., J. Immunol., 167:5185-192, 2001). ScFv dimers can also be formed through recombinant techniques known in the art. An example of the construction of scFv dimers is given in Goel et al. (Cancer Res. 60:6964-71, 2000). Antibody multimers may be purified using any suitable method known in the art, including, but not limited to, size exclusion chromatography.

Any of the antibodies or antigen-binding fragments described herein may be conjugated to a stabilizing molecule (e.g., a molecule that increases the half-life of the antibody or antigen-binding fragment thereof in a mammal or in solution). Non-limiting examples of stabilizing molecules include: a polymer (e.g., a polyethylene glycol) or a protein (e.g., serum albumin, such as human serum albumin) The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibody or antigen-binding antibody fragment binds to MT2 protein (e.g., extracellular human MT2 protein) and neutralizes a function of MT2 protein (e.g., prevents or decreases MT2 protein from binding to a TRPA1 receptor (e.g., a TRPA1 receptor on a fibroblast, a monocyte, or a neuron)).

### Aptamers

Additional examples of agents that bind to and/or neutralize a function of MT2 protein are aptamers. An aptamer is an oligonucleotide or peptide that is capable of binding to a specific polypeptide target. Methods of generating and screening oligonucleotide or peptide aptamers are known in the art. Exemplary methods for generating and screening oligonucleotide or peptide aptamers are described in U.S. Patent Application Publication Nos. 2012/0115752, and 2012/0014875; U.S. Pat. No. 7,745,607; and WO09/053691 (each of which is incorporated herein by reference). Additional methods for generating and screening oligonucleotide and peptide aptamers are described in Hoon et al., BioTechniques 51:413-416, 2011; Dausse et al., J. Nanobiotechnology 9:25, 2011; Hasegawa et al., Biotechnol. Lett. 30:829-834, 2008; and Drabovich et al., Analytical Chem. 78:6330-6335, 2006. Additional methods for generating and selecting aptamers that bind to and/or neutralize a function of MT2 protein (e.g., SEQ ID NO: 1) are known in the art.

Antibodies or Antigen-Binding Fragments that Specifically Bind to MT2 Protein

Antibodies or antigen-binding antibody fragments that specifically bind to MT2 protein can be generated using any of the techniques described above. As can be appreciated by those skilled in the art, an antibody that specifically binds to MT2 protein can be generated by immunizing an animal with an antigenic MT2 peptide that is unique or specific to MT2 protein (e.g., a peptide sequence present in a MT2 protein that is not present in a MT1 protein). For example, an antibody or antigen-binding antibody fragment that specifically binds to human MT2 protein can bind to an epitope containing all or a part of amino acids 8-17, amino acids 39-46, and/or amino acids 49-58 of SEQ ID NO: 1. In another example, an antibody or antigen-binding antibody fragment that specifically binds to rabbit MT2 protein can bind to an epitope (peptide) containing all or part of amino acids 9-24 (e.g., amino acids 9, 10, 12, 18, 21, and/or 24) and/or amino acids 40-43 (e.g., amino acids 40 and/or 43) of SEQ ID NO: 7. In another example, an antibody or antigen-binding antibody fragment that specifically binds to mouse MT2 protein can bind to an epitope containing all or part of amino acids 8-23 (e g , amino acids 8-10, 14, 16-18, 20, and/or 23), amino acids 42-25 (e.g., amino acids 42 and/or 45), and/or amino acids 49-58 (e g , amino acids 49, 52, 54, and/or 58) of SEQ ID NO: 9. Any of these exemplary epitopes can be used to generate an antibody or an antigen-binding antibody fragment that specifically binds to MT2 protein using the methods described herein or methods known in the art.

The antibodies or antigen-binding antibody fragments that specifically bind to MT2 protein can have an affinity for MT2 protein that is at least 10-fold (e.g., at least 15-fold, 20-fold, 25-fold, 30-fold, 35-fold, 40-fold, 45-fold, or 50-fold) greater than the antibody's or antigen-binding antibody fragment's affinity for MT1 protein. The antibody or antigen-binding antibody fragment that specifically binds to MT2 can be any of the exemplary antibodies or exemplary antigen-binding antibody fragments described herein.

Oligonucleotides that Decrease the Expression of MT2 mRNA in a Mammalian Cell

Non-limiting examples of oligonucleotides that can decrease the expression of MT2 mRNA in a mammalian fibrolast include inhibitory nucleic acids (e.g., small inhibitory nucleic acids (siRNA)), antisense oligonucleotides, and ribozymes. Exemplary aspects of these different oligonucleotides are described below.

Antisense Oligonucleotides

Oligonucleotides that decrease the expression of MT2 mRNA expression in a mammalian cell (e.g., a fibroblast) include antisense nucleic acid molecules, i.e., nucleic acid molecules whose nucleotide sequence is complementary to all or part of an mRNA based on the sequence of a gene encoding a MT2 protein (e.g., complementary to all or a part of SEQ ID NO: 2, 4, or 6). An antisense nucleic acid molecule can be antisense to all or part of a non-coding region of the coding strand of a nucleotide sequence encoding a MT2 protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense molecules to target a MT2 gene described herein. For example, a “gene walk” comprising a series of oligonucleotides of 15-30 nucleotides spanning the length of a MT2 gene can be prepared, followed by testing for inhibition of expression of the MT2 gene. Optionally, gaps of 5-10 nucleotides can be left between the oligonucleotides to reduce the number of oligonucleotides synthesized and tested.

An antisense oligonucleotide can be, for example, about 5, 10, 15, 20, 25, 30, 35, 40, 45, or 50 nucleotides or more in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosin, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules described herein can be prepared in vitro and administered to a mammal, e.g., a human. Alternatively, they can be generated in situ such that they hybridize with or bind to cellular mRNA and/or genomic DNA encoding a MT2 protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. For example, to achieve sufficient intracellular concentrations of the antisense molecules, vector constructs can be used in which the antisense nucleic acid molecule is placed under the control of a strong pol II or pol III promoter. In some embodiments, the vector used to express the oligonucleotide that decreases the expression of MT2 mRNA in a mammalian cell (e.g., a fibroblast) can be a lentivirus, a retrovirus, or an adenovirus vector.

An antisense nucleic acid molecule of the invention can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid molecule can also comprise a 2′-O-methylribonucleotide (Inoue et al., Nucleic Acids Res., 15:6131-6148, 1987) or a chimeric RNA-DNA analog (Inoue et al., FEBS Lett., 215:327-330, 1987).

Antisense molecules that are complementary to all or part of a MT2 gene are also useful for assaying expression of a MT2 gene using hybridization methods known in the art. For example, the antisense molecule is labeled (e.g., with a radioactive molecule) and an excess amount of the labeled antisense molecule is hybridized to an RNA sample. Unhybridized labeled antisense molecule is removed (e.g., by washing) and the amount of hybridized antisense molecule measured. The amount of hybridized molecule is measured and used to calculate the amount of expression of the MT2 mRNA. In general, antisense molecules used for this purpose can hybridize to a sequence from a MT2 gene under high stringency conditions such as those described herein. When the RNA sample is first used to synthesize cDNA, a sense molecule can be used. It is also possible to use a double-stranded molecule in such assays as long as the double-stranded molecule is adequately denatured prior to hybridization.

Ribozymes

Also provided are ribozymes that have specificity for sequences encoding an MT2 protein described herein (e.g., specificity for a MT2 mRNA, e.g., specificity for SEQ ID NO: 2, 4, or 6). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature, 334:585-591, 1988)) can be used to catalytically cleave mRNA transcripts to thereby inhibit translation of the protein encoded by the mRNA. A ribozyme having specificity for a nucleic acid molecule of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a MT2 mRNA (Cech et al. U.S. Pat. No. 4,987,071; and Cech et al., U.S. Pat. No. 5,116,742). Alternatively, an MT2 mRNA can be used to select a catalytic RNA having a specific ribonuclease activity from a pool of RNA molecules. See, e.g., Bartel and Szostak, Science, 261:1411-1418, 1993.

Also provided herein are nucleic acid molecules that form triple helical structures. For example, expression of a MT2 polypeptide can be inhibited by targeting nucleotide sequences complementary to the regulatory region of the gene encoding the MT2 polypeptide (e.g., the promoter and/or enhancer) to form triple helical structures that prevent transcription of the gene in target cells. See generally Helene, Anticancer Drug Des. 6(6):569-84, 1991; Helene, Ann. N.Y. Acad. Sci., 660:27-36, 1992; and Maher, Bioassays, 14(12):807-15, 1992.

In various embodiments, nucleic acid molecules (e.g., nucleic acid molecules used to decrease expression of MT2 mRNA in a mammalian cell) can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al., Bioorganic & Medicinal Chem., 4(1): 5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols, e.g., as described in Hyrup et al., 1996, supra; Perry-O'Keefe et al., Proc. Natl. Acad. Sci. USA, 93: 14670-675, 1996.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup, 1996, supra; or as probes or primers for DNA sequence and hybridization (Hyrup, 1996, supra; Perry-O'Keefe et al., Proc. Natl. Acad. Sci. USA, 93: 14670-675, 1996).

PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup,1996, supra). The synthesis of PNA-DNA chimeras can be performed as described in Hyrup,1996, supra, and Finn et al., Nucleic Acids Res., 24:3357-63, 1996. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., Nucleic Acids Res., 17:5973-88, 1989). PNA monomers are then coupled in a stepwise manner to produce a chimeric molecule with a 5′ PNA segment and a 3′ DNA segment (Finn et al., Nucleic Acids Res., 24:3357-63, 1996). Alternatively, chimeric molecules can be synthesized with a 5′ DNA segment and a 3′ PNA segment (Peterser et al., Bioorganic Med. Chem. Lett., 5:1119-11124, 1975).

In some embodiments, the oligonucleotide includes other appended groups such as peptides (e.g., for targeting host cell receptors in vivo), or agents facilitating transport across the cell membrane (see, e.g., Letsinger et al., Proc. Natl. Acad. Sci. USA, 86:6553-6556, 1989; Lemaitre et al., Proc. Natl. Acad. Sci. USA, 84:648-652, 1989; WO 88/09810) or the blood-brain barrier (see, e.g., WO 89/10134). In addition, oligonucleotides can be modified with hybridization-triggered cleavage agents (see, e.g., Krol et al., Bio/Techniques, 6:958-976, 1988) or intercalating agents (see, e.g., Zon, Pharm. Res., 5:539-549, 1988). To this end, the oligonucleotide may be conjugated to another molecule, e.g., a peptide, hybridization triggered cross-linking agent, transport agent, hybridization-triggered cleavage agent, etc.

siRNA

Another means by which expression of MT2 mRNA can be decreased in mammalian cells (e.g., mammalian fibroblasts) is by RNA interference (RNAi). RNAi is a process in which mRNA is degraded in host cells. To inhibit an mRNA, double-stranded RNA (dsRNA) corresponding to a portion of the gene to be silenced (e.g., a gene encoding a MT2 polypeptide) is introduced into a cell. The dsRNA is digested into 21-23 nucleotide-long duplexes called short interfering RNAs (or siRNAs), which bind to a nuclease complex to form what is known as the RNA-induced silencing complex (or RISC). The RISC targets the homologous transcript by base pairing interactions between one of the siRNA strands and the endogenous mRNA. It then cleaves the mRNA about 12 nucleotides from the 3′ terminus of the siRNA (see Sharp et al., Genes Dev. 15:485-490, 2001, and Hammond et al., Nature Rev. Gen., 2:110-119, 2001).

RNA-mediated gene silencing can be induced in mammalian cells in many ways, e.g., by enforcing endogenous expression of RNA hairpins (see, Paddison et al., Proc. Natl. Acad. Sci. USA, 99:1443-1448, 2002) or, as noted above, by transfection of small (21-23 nt) dsRNA (reviewed in Caplen, Trends in Biotech., 20:49-51, 2002). Methods for modulating gene expression with RNAi are described, e.g., in U.S. Pat. No. 6,506,559 and U.S. Patent Publication No. 2003/0056235, which are hereby incorporated by reference.

Standard molecular biology techniques can be used to generate siRNAs. Short interfering RNAs can be chemically synthesized, recombinantly produced, e.g., by expressing RNA from a template DNA, such as a plasmid, or obtained from commercial vendors such as Dharmacon. The RNA used to mediate RNAi can include synthetic or modified nucleotides, such as phosphorothioate nucleotides. Methods of transfecting cells with siRNA or with plasmids engineered to make siRNA are routine in the art.

The siRNA molecules used to decrease expression of a MT2 mRNA can vary in a number of ways. For example, they can include a 3′ hydroxyl group and strands of 21, 22, or 23 consecutive nucleotides. They can be blunt ended or include an overhanging end at either the 3′ end, the 5′ end, or both ends. For example, at least one strand of the RNA molecule can have a 3′ overhang from about 1 to about 6 nucleotides (e.g., 1-5, 1-3, 2-4 or 3-5 nucleotides (whether pyrimidine or purine nucleotides) in length. Where both strands include an overhang, the length of the overhangs may be the same or different for each strand.

To further enhance the stability of the RNA duplexes, the 3′ overhangs can be stabilized against degradation (by, e.g., including purine nucleotides, such as adenosine or guanosine nucleotides or replacing pyrimidine nucleotides by modified analogues (e.g., substitution of uridine 2 nucleotide 3′ overhangs by 2′-deoxythymidine is tolerated and does not affect the efficiency of RNAi). Any siRNA can be used in the methods of decreasing MT2 mRNA, provided it has sufficient homology to the target of interest (e.g., a sequence present in SEQ ID NO: 2, 4, or 6). There is no upper limit on the length of the siRNA that can be used (e.g., the siRNA can range from about 21 base pairs of the gene to the full length of the gene or more (e.g., 50-100, 100-250, 250-500, 500-1000, or over 1000 base pairs).

### Anti-Inflammatory Agents

In some instances, an anti-inflammatory agent can be admistered orally, intravenously, intraarterially, intrathecally, subcutaneously, or intramuscularly. Anti-inflammatory agents include, e.g., corticosteroids, non-steroidal anti-inflammatory drugs (NSAIDs, e.g., cyclooxygenase I (COX I) inhibitors and cyclooxygenase II (COX-II) inhibitors), immune selective anti-inflammatory derivatives (ImSAIDs), and biologics (e.g., an anti-TNFa antibody or antigen-binding antibody fragment, an anti-IL-1 antibody or antigen-binding antibody fragment, or an anti-NGF antibody or antigen-binding antibody fragment). Any of the exemplary anti-inflammatory agents described herein or known in the art can be included in any of the compositions described herein.

Non-limiting examples of NSAIDs that can be salicylates (e.g., aspirin, diflusinal, and salsalate), propionic acid derivatives (e.g., ibuprofen, dexiboprofen, naproxen, fenoprofen, ketoprofen, dexketoprofen, flurbiprofen, oxaprozin, and loxoprofen), acetic acid derivatives (e.g., indomethacin, sulindac, etodolac, ketorolac, diclofenac, and nabumetone), enolic acid derivatives (e.g., piroxicam, meloxicam, tanoxicam, droxicam, lornoxicam, and isoxicam), fenamic acid derivatives (e.g., mefamic acid, meclofenamic acid, flufenamic acid, and tolfenamic acid), sulphonanilides (e.g., nimesulide), licofelone, and lysine clonixinate. In some embodiments, an NSAID is a COX-I inhibitor or a COX-II inhibitor. Non-limiting examples of COX-I inhibitors include aspirin, ibuprofen, and naproxen. Non-limiting examples of COX-II inhibitors include celecoxib, valdecoxib, and rofecoxib.

Non-limiting examples of ImSAIDs include FEG (Phe-Glu-Gly), its D-isomer feG, and SGP-T peptide. Non-limiting examples of corticosteroids include hydrocortisone, cortisone acetate, tixocortol pivalate, prednisolone, methylprednisolone, prednisone, triamcinolone acetonide, triamcinolone alcohol, mometasone, amcinonide, budesonide, desonide, fluocinolone, halcinonide, betamethasone, dexamethasone, and fluocortolone. Non-limiting examples of biologics include tocilizumab, certolizumab, etanercept, adalimumab, anakinra, abatacept, efalizumab, infliximab, rituximab, golimumab, tanezumab, fulranumab, and REGN472.

### Analgesics

Analgescis are class of agents administered to treat pain in a mammal Any of the exemplary analgesics described herein or known in the art can be included in any of the compositions described herein. Non-limiting examples of analgesics include opioid drugs (e.g., morphine, opium, codeine, oxycodone, hydrocodone, diamorphine, dihydromorphine, pethidine, buprenorphine, fentanyl, methadone, meperidine, pentazocine, dipipanone,and tramadol), acetaminophen, venlafaxine, flupirtine, nefopam, gabapentin, pregabalin, orphenadrine, cyclobenzaprine, trazodone, clonidine, duloxetine and amitriptyline.

### Formulations and Dosages

Any of the compositions described herein can be a pharmaceutical composition. For example, a pharmaceutical composition containing an antibody or antigen-binding antibody fragment that specifically binds to MT2 protein, or a pharmaceutical composition containing an agent that binds to and/or neutralizes a function of MT2 protein and/or an oligonucleotide that decreases the expression of MT2 mRNA in a mammalian cell (e.g., a mammalian fibroblast) (and optionally at least one anti-inflammatory agent or analgesic), can contain one or more of: a pharmaceutically acceptable excipient or buffer, an antimicrobial or antifungal agent, or a stabilizing protein (e.g., human serum albumin)

Any of the compositions described herein can be formulated as a liquid for systemic administration. In some embodiments, the compositions are formulated for intraarterial, intravenous, intraperitoneal, intrathecal, ocular, nasal, intramuscular, intraductal, rectal, intravesical, or subcutaneous administration.

In some embodiments, the compositions are formulated as a solid. In some embodiments, the compositions are formulated for oral or topical (e.g., transdermal) administration. In some embodiments, the compositions are formulated as a suppository.

In some embodiments, the compositions are encapsulated in nanomaterials for targeted delivery (e.g., encapsulated in a nanomaterial having one or more tissue- or cell-targeting molecules on its surface). In some embodiments, the compositions are formulated as an emulsion or as a liposome-containing composition. In some embodiments, the compositions are formulated for sustained release (e.g., formulated in a biodegradable polymers or in nanoparticles). In some embodiments, the compositions are formulated in an implantable device that allows for sustained release of the agent that binds to and/or neutralizes a function of MT2 protein, the oligonucleotide that decreases the expression of MT2 mRNA in a mammalian cell (e.g., a mammalian fibroblast), an anti-inflammatory agent, and/or an analgesic. In some embodiments, the compositions are formulated in an implantable device that allows for sustained release of an antibody or an antigen-binding antibody fragment that specifically binds to MT2 protein.

Pharmaceutical compositions are formulated to be compatible with their intended route of administration or the intended target tissue, e.g., systemic or local administration. In some embodiments, the composition is delivered to an inflamed tissue in the mammal (e.g., by intramuscular, subcutaneous, intraperitoneal, intraarticular or intrathecal injection). In some embodiments, the compositions are formulated for oral, intravenous, intradermal, subcutaneous, transmucosal (e.g., nasal sprays are formulated for inhalation), or transdermal (e.g., topical ointments, salves, gels, patches, or creams as generally known in the art) administration. The compositions can include a sterile diluent (e.g., sterile water or saline), a fixed oil, polyethylene glycol, glycerine, propylene glycol, or other synthetic solvents; antibacterial or antifungal agents, such as benzyl alcohol or methyl parabens, chlorobutanol, phenol, ascorbic acid, thimerosal, and the like; antioxidants, such as ascorbic acid or sodium bisulfite; chelating agents, such as ethylenediaminetetraacetic acid; buffers such as acetates, citrates, or phosphates; and isotonic agents, such as sugars (e.g., dextrose), polyalcohols (e.g., manitol or sorbitol), or salts (e.g., sodium chloride). Liposomal suspensions can also be used as pharmaceutically acceptable carriers (see, e.g., U.S. Pat. No. 4,522,811; herein incorporated by reference). Preparations of the compositions can be formulated and enclosed in ampules, disposable syringes, or multiple dose vials that prevent exposure of the caged tamoxifen or caged tamoxifen derivative molecules to light. Where required (as in, for example, injectable formulations), proper fluidity can be maintained by, for example, the use of a coating such as lecithin, or a surfactant. Absorption of an agent that binds to and/or neutralizes a function of MT2 protein, an antibody or antigen-binding antibody fragment that specifically binds to MT2 protein, an oligonucleotide that decreases the expression of MT2 mRNA in a mammalian cell (e.g., a fibroblast), an anti-inflammatory agent, and/or an alagesic can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Where oral administration is intended, the agents can be included in pills, capsules, troches and the like, and can contain any of the following ingredients, or compounds of a similar nature: a binder, such as microcrystalline cellulose, gum tragacanth, or gelatin; an excipient, such as starch or lactose; a disintegrating agent, such as alginic acid, Primogel, or corn starch; a lubricant, such as magnesium stearate; a glidant, such as colloidal silicon dioxide; a sweetening agent, such as sucrose or saccharin; or a flavoring agent, such as peppermint, methyl salicylate, or orange flavoring.

The compositions described herein can be formulated for ocular or parenteral (e.g., oral) administration in dosage unit form (i.e., physically discrete units containing a predetermined quantity of active compound for ease of administration and uniformity of dosage). Toxicity and therapeutic efficacy of compositions can be determined by standard pharmaceutical procedures in cell cultures or experimental animals. One can, for example, determine the LD50 (the dose lethal to 50% of the population) and the ED50 (the dose therapeutically effective in 50% of the population), the therapeutic index being the ratio of LD50:ED50. Compositions that exhibit high therapeutic indices are preferred. Where a composition exhibits an undesirable side effect, care should be taken to target the composition to the site of the affected or targeted tissue (the aim being to minimize potential damage to unaffected cells and, thereby, reduce side effects). Toxicity and therapeutic efficacy can be determined by other standard pharmaceutical procedures.

In some embodiments, the compositions described herein are formulated in a single dosage form. In some embodiments, a single dosage of the composition contains between 1 mg to 500 mg, between 1 mg and 400 mg, between 1 mg and 300 mg, between 1 mg and 250 mg, between 1 mg and 200 mg, between 1 mg and 100 mg, and between 1 mg and 50 mg of an agent that binds to and/or neutralizes a function of MT2 protein and/or an antibody or antigen-binding antibody fragment that specifically binds to MT2 protein, and/or contains between 1 mg to 500 mg, between 1 mg and 400 mg, between 1 mg and 300 mg, between 1 mg and 250 mg, between 1 mg and 200 mg, between 1 mg and 100 mg, between 1 mg and 50 mg, between 1 mg and 10 mg of an oligonucleotide that decreases the expression of MT2 mRNA in a mammalian cell (e.g., a fibroblast).

In some embodiments, a single dosage of the composition contains between 1 mg to 500 mg, between 1 mg and 400 mg, between 1 mg and 300 mg, between 1 mg and 250 mg, between 1 mg and 200 mg, between 1 mg and 100 mg, and between 1 mg and 50 mg of an anti-inflammatory agent and/or between 1 mg to 500 mg, between 1 mg and 400 mg, between 1 mg and 300 mg, between 1 mg and 250 mg, between 1 mg and 200 mg, between 1 mg and 100 mg, and between 1 mg and 50 mg of an analgesic.

In some examples, the compositions (e.g., pharmaceutical compositions) contain at least one agent that binds to and/or neutralizes a function of a MT2 protein (e.g., any of the agents that bind to a MT2 protein described herein), antibody or antigen-binding antibody fragment that specifically binds to MT2 protein, and/or at least one oligonucleotide that decreases the expression of MT2 mRNA in a mammalian cell (e.g., a fibroblast) (e.g., any of the oligonucleotides that decrease the expression of MT2 mRNA in a mammalian cell (e.g., a fibroblast) described herein). These compositions can be formulated for any of the routes of administration described herein, using any of the formulations described herein, in any of the dosages described herein.

Also provided herein are kits that contain at least one dose of any of the compositions described herein. In some embodiments, the kits can further include an item for use in administering a composition (e.g., any of the compositions described herein) to the mammal (e.g., a syringe, e.g., a pre-filled syringe). In some embodiments, the kits contain one or more doses (e.g., at least two, three, four, five, six, seven, eight, nine, ten, eleven, twelve, thirteen, fourteen, twenty, thirty, or fourty doses) (e.g., oral doses) of any of the compositions described herein. In some embodiments, the kit further contains instructions for administering the composition (or a dose of the composition) to a mammal (e.g., a mammal having pain). In some embodiments, the kits contain a composition containing at least one agent that binds to and/or neutralizes a function of MT2 protein, an antibody or antigen- binding antibody fragment that specifically binds to MT2 protein, and/or an oligonucleotide that decreases the expression of MT2 mRNA in a mammalian cell (e.g., a fibroblast) (e.g., any of the agents or oligonucleotides described herein), and a composition containing at least one anti-inflammatory agent and/or analgesic (e.g., any of the anti-inflammatory agents and/or analgesics described herein). In some embodiments, the kit further contains instructions for performing any of the methods described herein.

### Methods of Treating Pain

Also provided are methods of decreasing pain (e.g., inflammatory pain, nociceptive pain, or neuropathic pain) in a mammal (e.g., a human) that include administering to the mammal an antibody or antigen-binding antibody fragment that specifically binds to MT2 protein or an agent that binds to and/or neutralizes a function of MT2 protein and/or an oligonucleotide that decreases the expression of MT2 mRNA in a mammalian cell (e.g., a fibroblast), in an amount sufficient to decrease the level of extracellular MT2 and/or neutralize a function of MT2 in the mammal, thereby decreasing pain in the mammal In some embodiments, the administering results in a decrease in mechanical sensitivity due to peripheral inflammatory pain in the mammal

In some embodiments, an agent that binds to and/or neutralizes a function of MT2 protein (e.g., an antibody or an antigen-binding antibody fragment that binds to and/or neutralizes a function of MT2, an antibody or antigen-binding antibody fragment that specifically binds to MT2 protein, or an aptamer) is administered to the mammal (e.g., any of the agents that bind to and/or neutralize a function of MT2 protein described herein). In some embodiments, an antibody or antigen-binding antibody fragment that binds to MT2 protein is administered to the mammal In some embodiments, the antibody that binds to MT2 protein is an IgG or an IgM antibody. In some embodiments, the antibody that binds to MT2 protein is a human or a humanized antibody. In some embodiments, the antigen-binding antibody fragment that binds to MT2 protein is a Fab fragment, a F(ab′)₂ fragment, a scFv fragment, or any of the other antigen-binding antibody fragments described herein. In some embodiments, the agent that binds to MT2 protein is an aptamer (e.g., a nucleic acid or peptide aptamer).

In some embodiments, the oligonucleotide that decreases the expression of MT2 mRNA in a mammalian cell (e.g., a fibroblast) is an inhibitory RNA (e.g., siRNA), an antisense oligonucleotide, or a ribozyme (e.g., any of the oligonucleotides that decrease the expression of MT2 mRNA in a mammalian cell (e.g., a fibroblast) described herein).

In some embodiments, the mammal (e.g., human) has been previously diagnosed as having pain (e.g., any of the different types of pain described herein, e.g., inflammatory pain, nociceptive pain, or neuropathic pain). A mammal having pain often experiences a dull, achy, stabbing, shooting, burning, or a pins-and-needles sensation. In some embodiments, the mammal may experience pain in a specific tissue of his or her body (i.e., localized pain). In some embodiments, the mammal can experience pain throughout his or her body. In some embodiments, the mammal can have acute pain (e.g., pain (e.g., consistent or recurrent) with a duration of less than a month). In some embodiments, the mammal can have chronic pain (e.g., pain (e.g., consistent or recurrent) that occurs for a month or more). In some embodiments, the pain is breakthrough pain.

In some embodiments, the subject can be diagnosed or suspected of having arthritis (e.g., rheumatoid arthritis or osteoarthritis), inflammatory bowel disease, urogenital pain, restless leg syndrome, polymyositis, postoperative and posttraumatic pain, pain due to a joint replacement, diabetic and HIV neuropathy, postherpetic neuralgia, pain due to cancer chemotherapeutic agensts, lumbar radiculopathy, and pancreatitis.

A mammal can be diagnosed as having pain by a medical or veterinary professional by interviewing (when the mammal is a human) and/or physically examining the mammal In some embodiments, a medical professional may diagnose a human as having pain by using, in part, a pain scale (e.g., Numeric Analog Scale from 0 to 10 (NAS-11), Faces Pain Scale-Revised, Wong-Baker FACES Pain Rating Scale, Coloured Analogue Scale, Visual Analog Scale, Brief Pain Inventory (BPI), Alder Hey Triage Pain Score, Behavioral Pain Scale (BPS), Checklist of Nonverbal Pain Indicators (CNPI), Critical-Care Pain Observation Tool (CPOT), COMFORT scale, Dallas Pain Questionnaire, Dolorimeter Pain Index (DPI), Face Legs Activity Cry Consolability Scale, Disease-Specific Pain Scale, Pediatric Pain Questionnaire (PPQ), Premature Infant Pain Profile (PIPP), Colorado Behavior Numerical Pain Scale, AUSCAN (e.g., used to assess hand osteoarthritis pain), and WOMAC (e.g., used to assess knee osteoarthritis pain). Additional methods of scoring pain are known in the art.

The efficacy of the treatment of pain can also be assessed by interviewing (when the mammal is a human) or physically examining the mammal (e.g., pain sensitivity or pain induced by movement). In some embodiments, any of the pain scores described above or known in the art can be used to determine the efficacy of the treatment of pain in the mammal For example, a successful treatment will result in a decrease in the pain score of a mammal

The mammal may be female or male, and may be an adult or juvenile (e.g., an infant). The mammal may have been previously treated with another analgesic or pain treatment and/or responded poorly to another pain treatment. The mammal may have tissue injury, a surgical wound, arthritis, or cancer. Where the mammal is an adult, the mammal may be, e.g., between 18 to 20 years old or at least or about 20, 25, 30, 35, 40, 45, 50, 55, 60, 65, 70, 75, 80, 85, 90, 95, or at least or about 100 years old).

The agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) may be administered by intravenous, intraarterial, subcutaneous, intraperitoneal, intramuscular, ocular, intraarticular, or intrathecal administration. In some instances, the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) is administered by local administration to an inflamed tissue or the locus of the pain in the mammal In other instances, the agent that binds to and/or neutralizes a function of MT2 protein, the antibody or antigen-binding antibody fragment that specifically binds to MT2 protein, and/or the oligonucleotide that decreases the expression of MT2 mRNA is systemically delivered to the mammal Combinations of such treatments are contemplated by the present invention.

The agent that binds to and/or neutralizes a function of MT2 protein, the antibody or antigen-binding antibody fragment that specifically binds to MT2 protein, and/or the oligonucleotide that decreases MT2 mRNA in a mammalian cell (e.g., a fibroblast) can be administered by a medical professional (e.g., a physician, a physician's assistant, a nurse, a nurse's assistant, or a laboratory technician) or veterinary professional. Alternatively or in addition, the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) can be self-administered by a human, e.g., the patient her/himself. The agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) can be administered in a hospital, a clinic, or a primary care facility (e.g., a nursing home), or any combination thereof.

The appropriate amount (dosage) of the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) administered can be determined by a medical professional or a veterinary professional based on a number of factors including, but not limited to, the type of pain (e.g., inflammatory pain, nociceptive pain, or neuropathic pain, and whether the pain is localized or systemic), the route of administration, the severity of pain (e.g., assessed using any of the pain scores described herein), the mammal's responsiveness to other pain treatments, the health of the mammal, the mammal's mass, the other therapies administered to the mammal, the age of the mammal, the sex of the mammal, and any other co-morbidity present in the mammal

A medical professional or veterinary professional having ordinary skill in the art can readily determine the effective amount of the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) that is required. For example, a physician or veterinarian could start with doses of the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) (e.g., any of the agents that bind to and/or neutralize a function of MT2 protein, the antibodies or antigen-binding antibody fragments that specifically bind to MT2 protein, and/or any of the oligonucleotides that decrease the expression of MT2 mRNA in a mammalian cell (e.g., a fibroblast) described herein) at levels lower than that required to achieve the desired therapeutic effect and then gradually increase the dose until the desired effect is achieved.

In some embodiments, the mammal is administered a dose of between 1 mg to 500 mg each of any of the agents that bind to and/or neutralize a function of MT2 protein and/or any of the antibodies or antigen-binding antibody fragments that specifically bind to MT2 protein described herein (e.g., between 1 mg to 400 mg, between 1 mg to 300 mg, between 1 mg and 250 mg, between 1 mg and 200 mg, between 1 mg and 150 mg, between 1 mg and 100 mg, between 1 mg and 50 mg, between 5 mg and 50 mg, and between 5 mg and 40 mg). In some embodiments, the mammal is administered a dose of between 1 mg to 500 mg of any of the oligonucleotides that decrease the expression of MT2 mRNA in a mammalian cell (e.g., a fibroblast) described herein (e.g., between 1 mg to 400 mg, between 1 mg to 300 mg, between 1 mg and 250 mg, between 1 mg and 200 mg, between 1 mg and 150 mg, between 1 mg and 100 mg, between 1 mg and 50 mg, between 5 mg and 50 mg, and between 5 mg and 40 mg).

In some embodiments, the mammal is further administered an anti-inflammatory agent (e.g., any of the anti-inflammatory agents described herein) and/or an analgesic (e.g., any of the analgesics described herein). In some embodiments, the mammal is administered a dose of between 1 mg to 500 mg of any of the anti-inflammatory agents and/or analgesics described herein (e.g., between 1 mg to 400 mg, between 1 mg to 300 mg, between 1 mg and 250 mg, between 1 mg and 200 mg, between 1 mg and 150 mg, between 1 mg and 100 mg, between 1 mg and 50 mg, between 5 mg and 50 mg, and between 5 mg and 40 mg). The anti-inflammatory agent and/or the analgesic can be administered to the mammal at substantially the same time as the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast). Alternatively or in addition, the anti-inflammator agent and/or the analgesic may be administered to the mammal one or more time points other than the time point at which the agent that binds to and/or neutralizes a function of MT2 protein, the antibody or antigen-binding antibody fragment that specifically binds to MT2 protein, and/or the oligonucleotide that descreases expression of MT2 mRNA is administered. In some embodiments, the anti-inflammatory agent and/or the analgesic is formulated together with an agent that binds to and/or neutralizes a function of MT2 protein, an antibody or antigen-binding antibody fragment that specifically binds to MT2 protein, and/or an oligonucleotide that decreases the expression of MT2 mRNA in a mammalian cell (e.g., a fibroblast) (e.g., using any of the exemplary formulations and compositions described herein). In some embodiments, the anti-inflammatory agent and/or the analgesic are formulated in a first dosage form, and the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) is formulated in a second dosage form. In some embodiments where the anti-inflammatory agent and/or the analgesic are formulated in a first dosage form, and the agent that binds to and/or neutralizes a function of MT2 protein, the antibody or antigen-binding antibody fragment that specifically binds to MT2 protein, and/or the oligonucleotide that decreases the expression of MT2 mRNA is formulated in a second dosage form, the first dosage form and the second dosage form can be formulated for the same route of administration (e.g., oral, subcutaneous, intramuscular, intravenous, intaarterial, intrathecal, and intraperitoneal administration) or can be formulated for different routes of administration (e.g., the first dosage form formulated for oral administration and the second dosage form formulated for subcutaneous administration). Combinations of such treatment regimes are clearly contemplated in the present invention.

The amount of the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) (and optionally, an anti-inflammatory agent and/or analgesic) administered will depend on whether the administration is local or systemic. In some embodiments, the mammal is administered more than one dose of the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast). In some embodiments, the mammal is administered more than one dose of any of the compositions described herein. In some embodiments, the mammal is administered a dose of an agent that binds to and/or neutralizes a function of MT2 protein, an antibody or antigen-binding antibody fragment that specifically binds to MT2 protein, and/or an oligonucleotide that decreases the expression of MT2 mRNA in a mammalian cell (e.g., a fibroblast) at least once every three months month (e.g., at least once every two months, at least once a month, at least twice a month, at least three times a month, at least four times a month, at least once a week, at least twice a week, three times a week, once a day, or twice a day).

In some embodiments, an agent that binds to and/or neutralizes a function of MT2 protein, an antibody or antigen-binding antibody fragment that specifically binds to MT2 protein, and/or an oligonucleotide that decreases the expression of MT2 mRNA in a mammalian cell (e.g., a fibroblast) is administered to a mammal chronically. In some embodiments, any of the compositions described herein is administered to the mammal chronically. Chronic treatments include any form of repeated administration for an extended period of time, such as repeated administrations for one or more months, between a month and a year, one or more years, or longer. In some embodiments, chronic treatments can involve regular administrations, for example one or more times a day, one or more times a week, or one or more times a month. In general, a suitable dose such as a daily dose of the agent that binds to and/or neutralizes a function of MT2 protein, the antibody or antigen- binding antibody fragment that specifically binds to MT2 protein, and/or the oligonucleotide that decreases the expression of MT2 mRNA in a mammalian cell (e.g., a fibroblast) will be the amount of the agent and/or oligonucleotide that is the lowest dose effective to produce a desired therapeutic effect. Such an effective dose will generally depend upon the factors described herein. If desired, the effective daily dose of the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) can be administered as two, three, four, five, or six or more sub-doses administered separately at appropriate intervals throughout the day, optionally, in unit dosage forms.

In some embodiments, the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) is formulated for sustained-release (e.g., formulated in a biodegradable polymer or a nanoparticle). In some embodiments, the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) is administered locally to the site of pain in the mammal In some embodiments, the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) is administered systemically (e.g., oral, intravenous, intaarterial, intraperitoneal, intramuscular, or subcutaneous administration). In some embodiments, the agent that binds to and/or neutralizes a function of MT2 protein, the antibody or antigen-binding antibody fragment that specifically binds to MT2 protein, and/or the oligonucleotide that decreases the expression of MT2 mRNA in a mammalian cell (e.g., a fibroblast) is formulated for oral, intraglandular, periglandular, subcutaneous, interductal, intramuscular, intraperitoneal, intraarticular, rectal, epidural, intraarterial, transdermal, or intravenous administration.

### Screening Methods

Also provided herein are methods of identifying a candidate agent that decreases pain (e.g., inflammatory pain, nociceptive pain, or neuropathic pain) in a mammal that include providing a mammalian (e.g., human) cell that expresses MT2 (e.g., fibroblast, a neutrophil, a T-cell, and a glial cell), contacting the mammalian cell with a candidate agent, determining a test level of MT2 expression (e.g., MT2 protein or MT2 mRNA) by the mammalian cell, comparing the test level of MT2 expression by the mammalian cell to a reference level of MT2 expression (e.g., MT2 protein or MT2 mRNA) in a control mammalian cell that expresses MT2 untreated with the candidate agent, and selecting a candidate agent that results in a test level of MT2 expression that is lower than the reference level of MT2 expression as being useful for decreasing pain in a mammal

In some embodiments, the mammalian (e.g., human) cell that expresses MT2 (e.g., fibroblast) is in vitro. Some embodiments where the mammalian cell (e.g., fibroblast) is in vitro further include administering the selected candidate agent to an animal model of pain (e.g., any of the animal models of pain described herein or known in the art). Forty different animal models of neuropathic pain are described in Jaggi et al. (Fundamental Clin. Pharmacol. 25:1-28, 2011). Different animal models of inflammatory pain are described in Ren et al. (ILAR Journal vol. 40, 1999). Additional animal models of pain are described in Ma and Zhang, Animal Models of Pain, Springer Protocols, Humana Press, 2010.

In some embodiments, the mammalian cell (e.g., fibroblast) is in a mammal, and the contacting is performed by administering the candidate agent to the mammal (e.g., by oral, subcutaneous, intravenous, intraarterial, intraperitoneal, intramuscular, or intrathecal administration).

In some embodiments, the test level and the reference level of MT2 expression is a level of extracellular MT2 protein (e.g., SEQ ID NO: 1). In some embodiments, the test level and the reference level of MT2 expression is a level of intracellular MT2 protein (e.g., SEQ ID NO: 1). In some embodiments, the test level and the reference level of MT2 expression is a level of MT2 mRNA (mRNA encoding MT2 protein, e.g., SEQ ID NO: 2).

In some embodiments, the reference level of MT2 expression is a level of MT2 expression of a control, in vitro, mammalian cell (e.g., fibroblast) that expresses MT2 untreated with the candidate agent. In some embodiments, the reference level of MT2 expression is a level of MT2 expression of a control in vivo mammalian cell (e.g., fibroblast) that expresses MT2 untreated with the candidate agent.

Methods for determining the level of MT2 protein expression (e.g., intracellular and extracellular MT2 protein) are known in the art. For example, levels of MT2 protein expression can be determined using an antibody or an antigen-binding antibody fragment that binds to MT2 protein (e.g., any of the antibodies or antigen-binding antibody fragments described herein). In some embodiments, the amount of MT2 protein expression can be determined using an antibody or antigen-binding antibody fragment that binds to MT2 in an enzyme-linked immunosorbent assay (ELISA). Antibodies that bind to MT2 protein are commercially available (see, the exemplary antibodies that bind to MT2 described herein).

Methods for determining the level of MT2 mRNA expression are also known in the art. For example, levels of MT2 mRNA expression can be determined using polymerase chain reaction (PCR) techniques, including reverse transcriptase (RT)-PCR and real-time RT- PCR using primers that are complementary to a MT2 mRNA (see, e.g., the exemplary MT2 mRNAs described herein, e.g., SEQ ID NO: 2). Additional sequences for mammalian MT2 mRNAs are known in the art.

Some embodiments of these methods further include generating a pharmaceutical composition for treating pain that includes the candidate agent.

The invention is further described in the following example, which does not limit the scope of the invention described in the claims.

### EXAMPLES

### Example 1

### MT2 is an Agonist of TRPA1 Activity

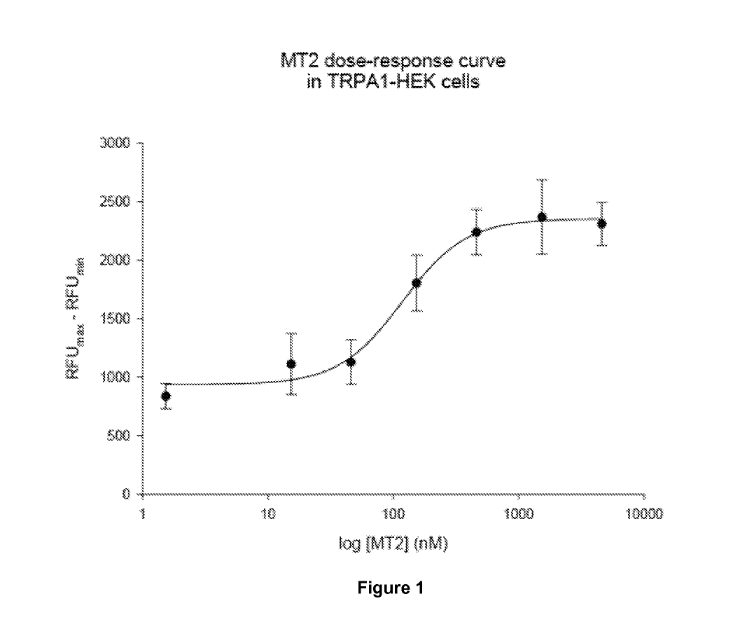

TRPA1 is a cellular receptor known to detect exogenous and endogenous damaging stimuli, and play a role in thermal hyperalgesia and mechanical allodynia. An experiment was performed to test whether a number of different proteins would stimulate calcium flux in human embryonic kidney (HEK) cells transfected to express human TRPA1 (hTRPA1). The proteins tested included CX3CL1, G-CSF, INF-γ, IL-1β, IL-3, IL-4, IL-5, IP-10, ITAC, KGF, MCP-1, MT1, MT2, NGF, PDGF, SLPI, Substance P, and TGF-β. In these experiments, mustard oil (MO) was used as a positive control. The protein that induced the greatest amount of calcium flux in the hTRPA1-transfected HEK cells was MT2. The data show that MT2 stimulates calcium flux with an EC₅₀ of ˜200 nM (FIG. 1).

### Example 2

### MT2 Expression is Induced in a Mouse Inflammation Model

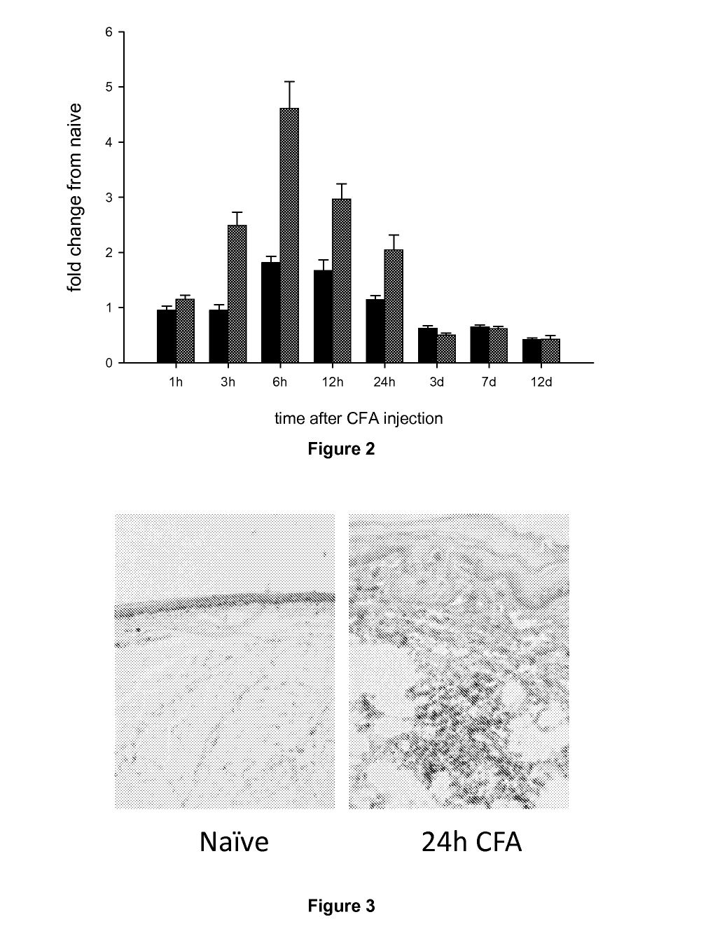

Additional experiments were performed to determine whether MT2 expression is induced during inflammation. In these experiments, mice were intraplantarly injected with Freund's adjuvant (CFA), and the expression of both MT1 and MT2 mRNA was assessed at different time points following the CFA injection. The data show that both MT1 and MT2 mRNA expression was increased at 3 hours post-injection in the hindpaw, with MT2 mRNA showing a higher level of induction (FIG. 2). Histological analysis of the hindpaw from a control mouse and a mouse 24-hours after CFA injection are shown in FIG. 3. The images in FIG. 3 show that, in the CFA-injected mouse, MT1/2 is expressed in basal keratinocytes, fibroblasts, and some macrophages at the site of injection.

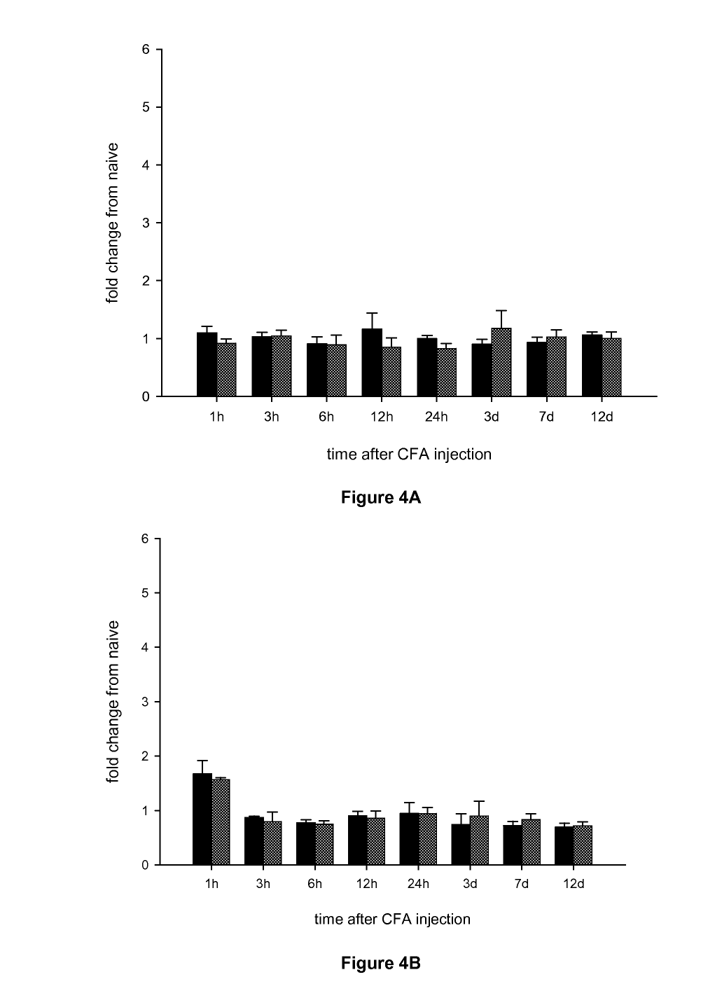

The expression of MT1 and MT2 mRNA was also assessed in the spinal cord and dorsal root ganglion in mice at different time points following CFA injection (FIGS. 4A and 4B). The data in FIGS. 4A and 4B show that MT1 and MT2 mRNA were not induced in either the spinal cord or the dorsal root ganglion following CFA injection.

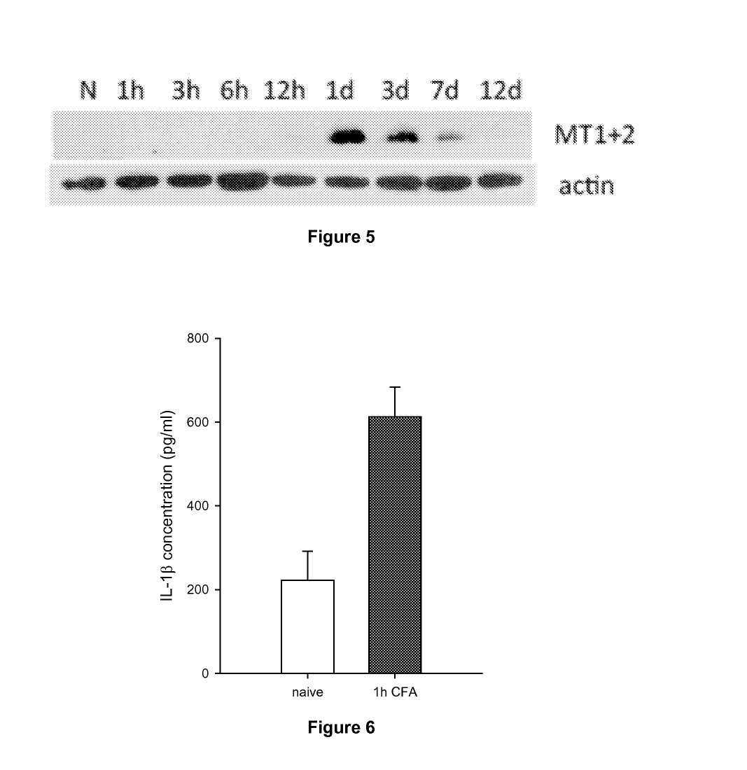

MT1/2 protein expression was also induced in the hindpaw of mice following injection with CFA (FIG. 5). These data indicate that the expression of MT2 is increased in inflamed tissues.

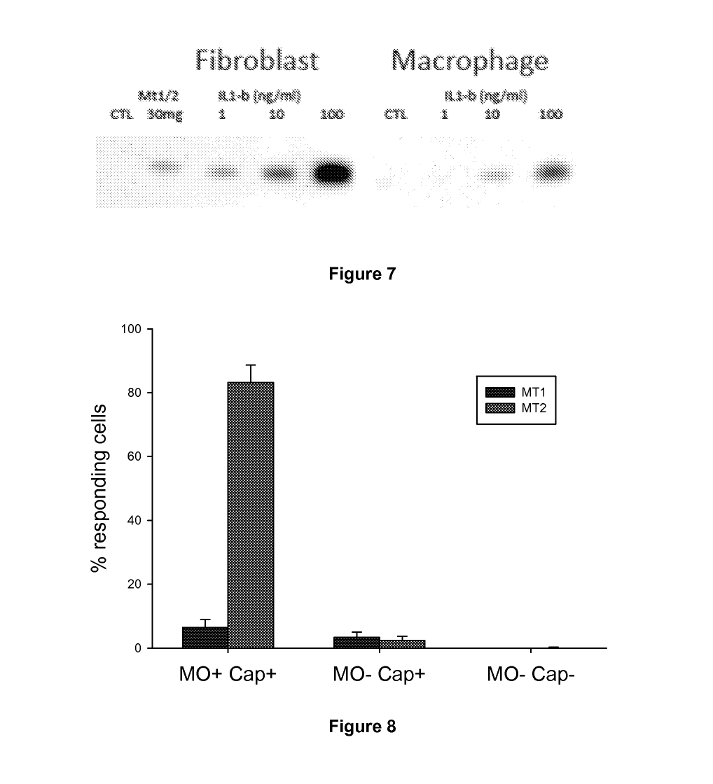

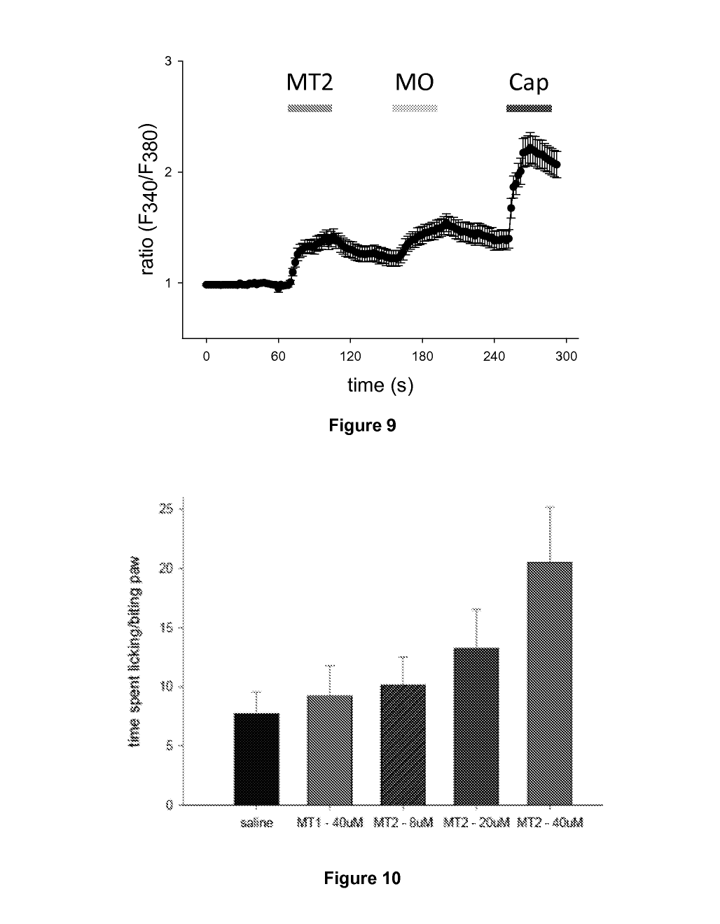

In order to further determine what cytokine induces expression of MT2 in inflamed tissues, the level of IL-1β in control mice and mice injected with CFA (at 1 hour after injection) was measured. The data show that IL-1β is significantly increased in mice 1-hour after CFA injection (FIG. 6). An additional experiment was performed to determine whether IL-1β can induce MT1/2 secretion in fibroblasts or macrophages. The data from these experiments show that IL-1β stimulates MT1/2 secretion in both fibroblasts and macrophages (FIG. 7).

### Example 3

### MT2 Stimulates Calcium Influx in Sensory Neurons

An additional set of experiments was performed to determine whether MT2 would have a physiological effect on sensory neurons. In these experiments, different subsets of sensory neurons from dorsal root ganglia (TRPA1+/TRPV1+, TRPA1−/TRPV1+, TRPA1−/TRPV1−) were treated with either 152 nM MT1 or 152 nM MT2, and the percentage of cells with stimulated calcium flux was determined as generally described in Binshtok et al. (Anesthesiology 111(1):127-137, 2009) and Grynkiewicz et al. (J. Biol. Chem. 260(6):3440-3450, 1985). The data from these experiments show that MT2 stimulates calcium flux in MO⁺Cap⁺ sensory neurons (FIG. 8, 9). The MT 1 and MT2 used in these experiments are bound to 7 zinc atoms each. While these two MTs have similar release kinetics for zinc (Chen et al., Biochemistry 41(26):8360-8367, 2002), only MT2 shows an effect on TRPA1+/TRPV1+sensory neurons which would rule out any effect for zinc.

### Example 4

### MT2 Induces Pain in a Mouse Model

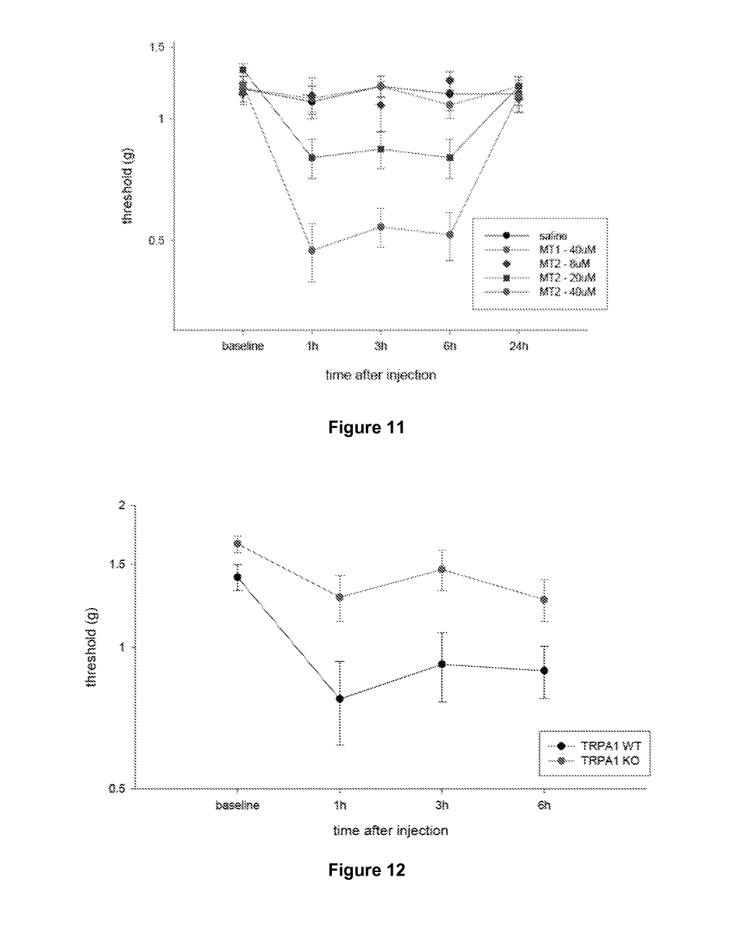

Additional experiments were performed to determine whether injection of MT2 would induce acute pain in a mouse model. In these experiments, mice were injected with saline, 40 μM MT1, 8 μM MT2, 20 μM MT2, or 40 μM MT2, and the amount of acute pain experienced by these mice was assessed by measuring the amount of time the mouse spent licking or biting the injected paw (over the first 20 minutes) (general pain assay generally performed as described in Boyce-Rustay et al., Methods Mol. Biol. 617:41-55, 2010). The data from this experiment shows that MT2 increases acute pain in the mice (FIG. 10). A similar experiment was performed, except pain in the mice was assessed by determining the mechanical threshold needed to elicit a 50% response in the mice (mechanical hypersensitivity assay performed as generally described in Cobos et al., Pain 153(4):876-884, 2012). The data from these experiments show that MT2 increases mechanical hypersensitivity in mice (FIG. 11).

Experiments were also performed to determine whether MT2 was inducing pain through transient receptor potential cation channel, member A1 (TRPA1) (a cation channel), a receptor known as a sensor for pain (e.g., inflammatory pain). In these experiments, wild type and TRAP1 knockout littermate mice were administered 40 μM MT2, and the mechanical threshold required to elicit a 50% response in the mice (mechanical hypersensitivity) was assessed in the mice. The data show that TRPA1 knockout mice demonstrated less mechanical hypersentivity following treatment with MT2, compared to the wild type mice receiving the same treatment (FIG. 12). These data indicate that MT2 is inducing pain through the TRPA1 receptor.

### Example 5

### MT2 Induces a Current Response in TRPA1-Expressing HEK Cells

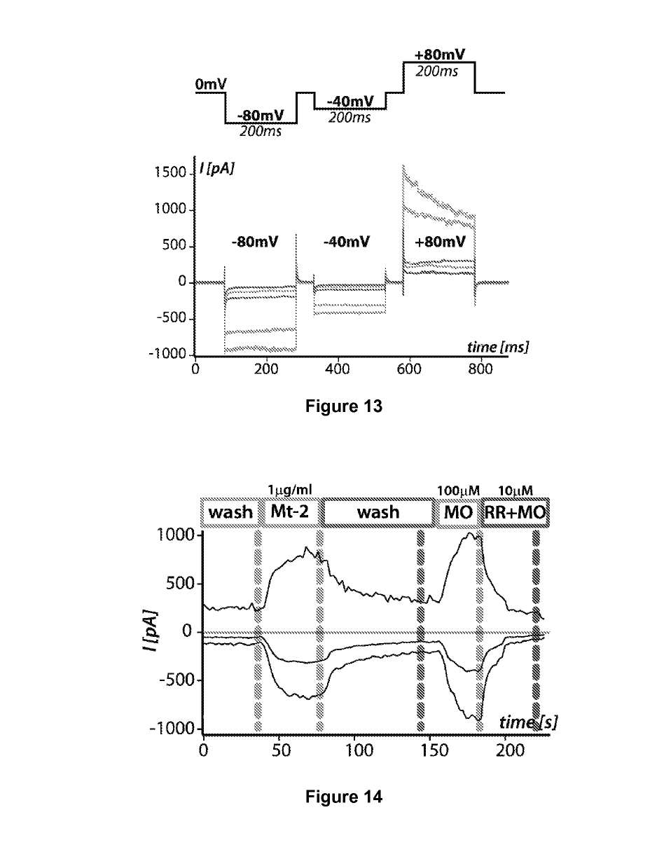

A set of experiments was performed to determine whether MT2 would induce a change in the current of TRPA1-expressing HEK cells. Whole cell patch-clamp recordings were made from HEK cells transfected with hTRPA1 in voltage-clamp mode. Patch pipettes were fabricated from thin-walled, borosilicate glass-capillary tubing (1 5 mm outer diameter; World Precision Instruments). Extracellular saline solution contained the following: 145 mM NaCl, 5 mM KCl, 2 mM CaCl₂, 1 mM MgCl₂, 10 mM glucose, 10 mM HEPES, pH 7.4. The same solution was used for washing and to dissolve MT2, mustard oil, and ruthenium red. The internal pipette solution contained the following: 145 mM KCl, 1 mM MgCl₂, 10 mM HEPES, 5 mM EGTA, pH 7.3. The resistance of a typical patch pipette was 3-4 MΩ. Membrane currents were amplified with an Axopatch 200B amplifier (Molecular Devices) in voltage-clamp mode. Signals were filtered at 2 kHz and digitized at 20 kHz. Data were stored with a personal computer using pCLAMP 10 software (Molecular Devices) and analyzed with ClampFit (Molecular Devices). After establishing the whole-cell configuration, cells were held at 0 mV. The cells were stimulated with a voltage protocol indicated in the upper panel of FIG. 13 (100 ms at 0 mV, 200 ms at −80 mV, 50 ms at 0 mV, 200 ms at −40 mV, 50 ms at 0 mV, 200 ms at +80mV, 100 ms at 0 mV) every 2 seconds during the entire time course of the experiment to monitor the current response under different conditions. The current responses to washes, MT2, mustard oil, and ruthenium red are shown in the lower panel of FIG. 13. To analyze the current response over a whole experiment, the last 50 ms of each voltage step (−80, −40, +80 mV) were averaged and plotted over the time course of the experiment in FIG. 14. The thick vertical lines indiciate the time points of individual traces for FIG. 13. The application of MT2 to human embryonic kidney (HEK) cells transfected with hTRPA1 leads to an inward and outward current, which is slighty outwardly rectifying , consistent with the current profile of hTRPA1.

### Example 6

### MT1/2 Knockout Mice Demonstrate Pain Resistance

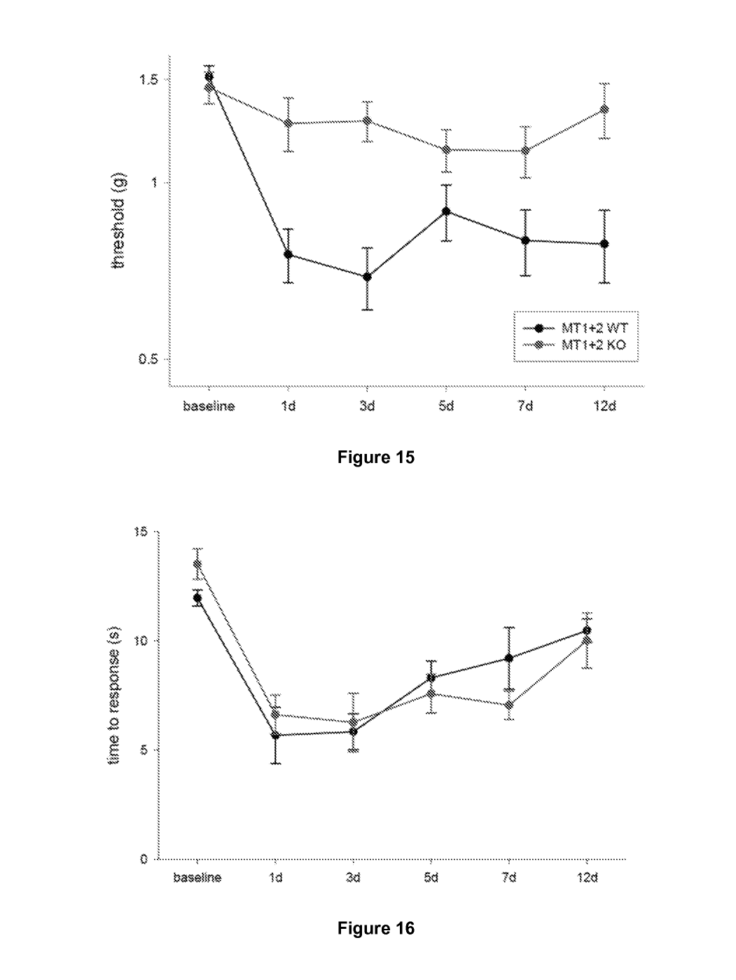

The mechanical hypersensitivity and heat hypersentivity in wild type and MT1/2 knockout mice were tested following CFA injection in the hindpaw (20 μL CFA injected into the left hindpaw using a 26 g Hamilton syringe). Mechanical hypersensitivity was tested using the methods described above. Heat hypersensitivity was determined using the methods described in Hargreaves et al., Pain 32(1):77-88, 1998. The resulting data show that MT1/2 knockout mice show less mechanical hypersensitity and do not show a significant change in radiant heat hypersensitivity, compared to wild type mice (see, FIGS. 15 and 16, respectively).

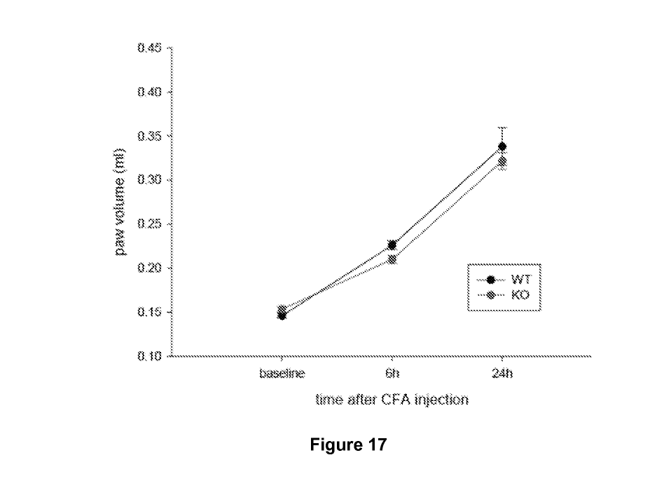

An additional set of experiments was performed to determine whether MT1/2 modulate the immune response in the wild type or knockout mice. In a first experiment, the paw size of both wild type and MT 1/2 knockout mice was measured both before, 6-hours after, and 24-hours after CFA injection. Paw size following CFA injection is an indirect measure of inflammation and edema in the mice. The data from this experiment show that there is no significant difference in paw size over time between the wild type and MT1/2 knockout mice (FIG. 17) and therefore, MT2 does not mediate a significant increase/decrease in inflammation (within the first 24 hours).

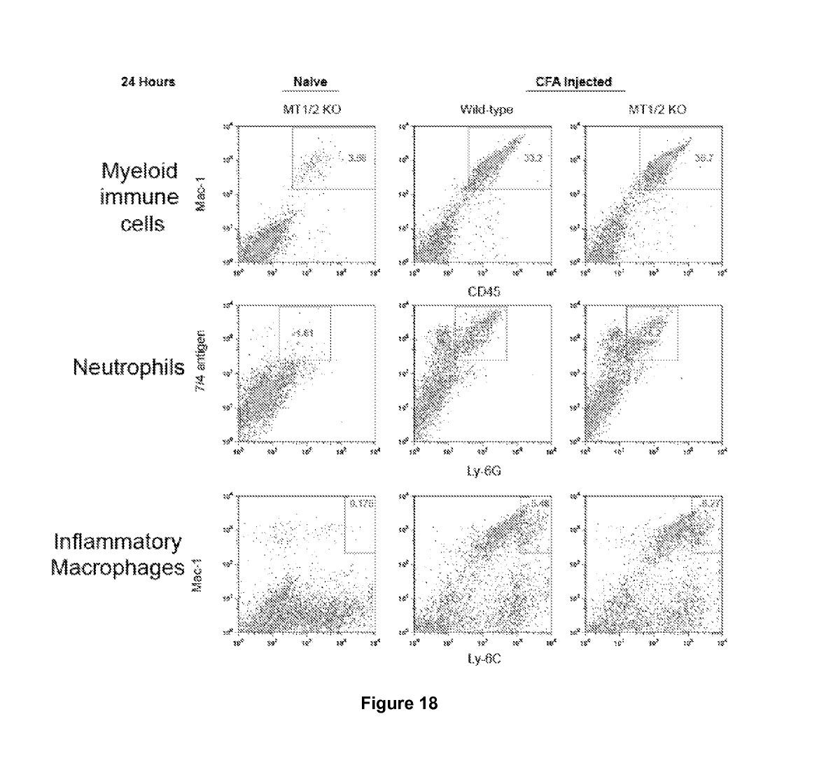

Experiments were performed to determine whether there was a difference in the populations of different immune cells in the wild-type and MT1/2 knockout mice. In these experiments, fluorescence assisted cell sorting was used to determine the percentage of myeloid immune cells, neutrophils, and inflammatory macrophages present in naïve MT1/2 knockout mice, wild type mice 24-hours after injection with CFA, and MT1/2 knockout mice 24-hours after injection with CFA. The data show that there was no significant difference in the myeloid immune cell, neutrophil, and inflammatory macrophage populations between the CFA-injected wild type mice and the CFA-injected MT1/2 knockout mice (FIG. 18).

### Example 7

### Antibodies that Bind MT2 Can Decrease Mechanical Hypersensitivity

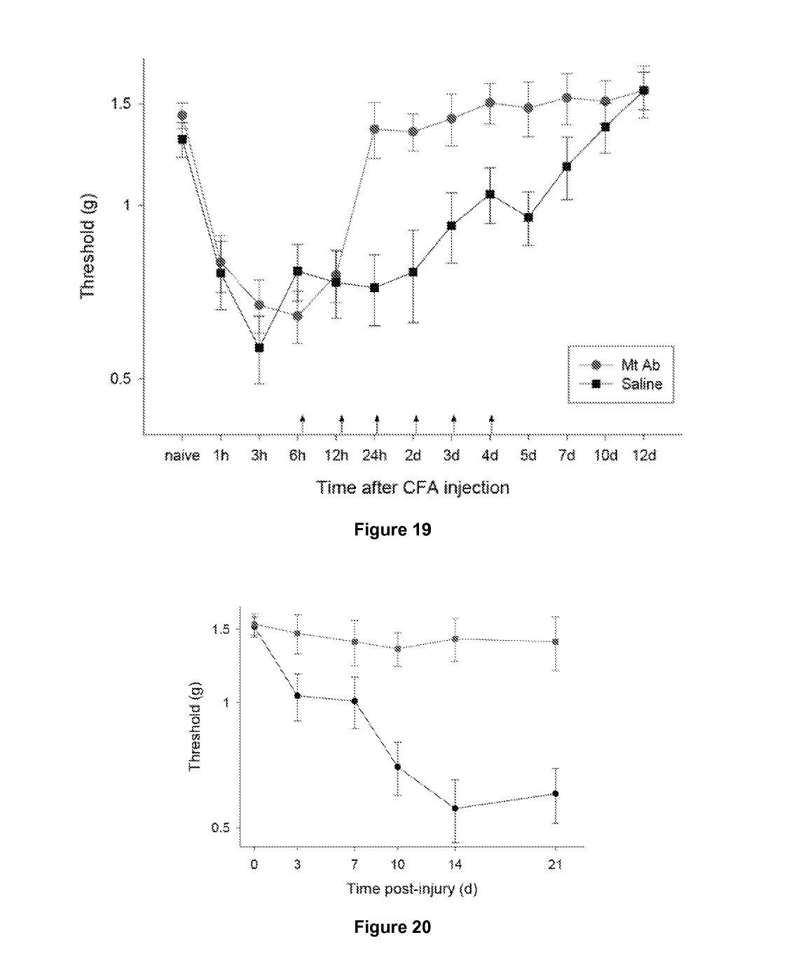

A set of experiments was performed to determine whether administration of an antibody that binds to MT2 would be able to decrease pain (as measured in this instance by mechanical hypersensitivity) in wild type mice injected with CFA. In these experiments, mice were injected at t=0 with CFA, and then subsequently administered either saline or 10 μL of anti-MT1/2 antibody (Dako; 130 mg/L) at 6 hours, 12 hours, 24 hours, 2 days, 3 days, and 4 days following injection of CFA. The data show that mice injected with the anti-MT1/2 antibody have decreased mechanical hypersensitivity compared to mice injected with saline (FIG. 19). These data indicate that agents that bind MT2 can be used to decrease pain in a mammal

### Example 8

### MT2 Plays a Role in Neuropathic Pain

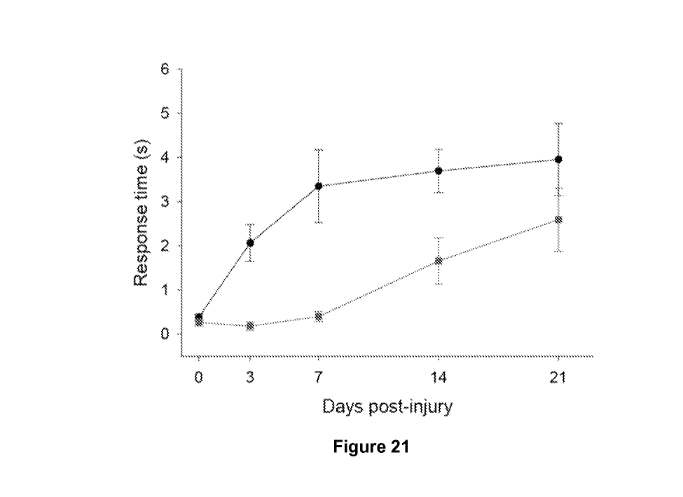

Experiments were performed using a spared nerve injury model of neuropathic pain in both wild type and MT1/2 knockout mice, and the mechanical thresholds needed to elicit a 50% response and cold response using the acetone test were assessed. The data from these experiments show that MT1/2 knockout mice have an absence of mechanical hypersensitivity for the duration of the experiment (21 days) and a lack of cold hypersensitivity in the first two weeks after injury (see, FIGS. 20 and 21, respectively). These data indicate that MT2 also plays a role in neuropathic pain, and that agents that specifically bind to MT2 may also decrease neuropathic pain in a mammal

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
an agent that binds to metallothionein 2 (MT2) protein or an oligonucleotide that decreases the expression of MT2 mRNA in a mammalian cell; and
one or more anti-inflammatory agents or analgesics.

2. The composition of claim 1, wherein the one or more anti-inflammatory agents are selected from the group consisting of: a non-steroidal anti-inflammatory drug (NSAID), a corticosteroid, an immune selective anti-inflammatory derivative (ImSAID), and a biologic.

3. The composition of claim 2, wherein the biologic is an anti-tumor necrosis factor a antibody, an anti-interleukin-1 antibody, or an anti-nerve growth factor antibody.

4. The composition of claim 2, wherein the NSAID is a cyclooxygenase-I (COX-1) inhibitor or a COX-II inhibitor.

5. The composition of claim 1, wherein the agent is an antibody or an antigen- binding antibody fragment that binds to MT2 protein.

6. The composition of claim 5, wherein the antigen-binding antibody fragment is selected from the group consisting of: a Fab fragment, a F(ab′)₂ fragment, and a scFv fragment.

7. The composition of claim 5, wherein the antibody is a humanized, an IgG, or an IgM antibody.

8.-11. (canceled)

12. A method of decreasing pain in a mammal, the method comprising administering to the mammal an agent that binds to metallothionein 2 (MT2) protein or an oligonucleotide that decreases the expression of MT2 mRNA in a mammalian cell, in an amount sufficient to decrease the level of extracellular MT2 protein or neutralize a function of extracellular MT2 protein in the mammal, thereby decreasing pain in the mammal.

13. The method of claim 12, wherein the pain is inflammatory pain or neuropathic pain.

14. (canceled)

15. The method of claim 12, wherein the agent is an antibody or an antigen- binding antibody fragment that binds to MT2 protein.

16. The method of claim 15, wherein the antigen-binding antibody fragment is selected from the group consisting of: a Fab fragment, a F(ab′)₂ fragment, and a scFv fragment.

17. The method of claim 15, wherein the antibody is a humanized. an IgG, or an IgM antibody.

18.-21. (canceled)

22. The method of claim 12, wherein the mammal has been diagnosed as having pain.

23. The method of claim 12, wherein the mammal is a human.

24. The method of claim 12, wherein the administering is performed by intravenous, intraarterial, subcutaneously, intramuscular, intraarticular, epidural, intrathecal, or intraperitoneal injection.

25. The method of claim 12, wherein the agent is administered to the mammal at least once every three months.

26. The method of claim 25, wherein the agent is administered to the mammal at least once a week.

27. The method of claim 12, wherein the mammal is administered at least one additional anti-inflammatory agent or analgesic.

28.-34. (canceled)


SEQUENCE LISTING (12 sequences)

SEQ ID NO: 1 - protein, 61 aa, homo sapiens
MDPNCSCAAGDSCTCAGSCKCKECKCTSCKKSCCSCCPVGCAKCAQGCICKGASDKCSCCA

SEQ ID NO: 2 - DNA, 327 bp, homo sapiens
atggaccccaactgctcgtgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctgatgctgggacagcccgctcccagatgtaaagaacgcgacttccacaaacctggattttttatgtacaaccctgaccgtgaccgtttgctatattcctttttctatgaaataatgtgaatgataataaaacagctttgtcttg

SEQ ID NO: 3 - protein, 61 aa, canis lupus
MDPNCSCAAGGSCTCAGSCKCKECRCTSCKKSCCSCCPVGCAKCAQGCICKGASDKCSCCA

SEQ ID NO: 4 - DNA, 430 bp, canis lupus
agcgccgccgtctctccgcccgctgtcccggactccagccgccccttctcgccatggatcccaactgctcctgcgccgcggggggctcctgcacgtgcgccggctcctgcaaatgcaaagagtgcagatgcacctcctgcaagaagagctgctgctcctgctgccccgtgggctgtgccaagtgtgcccagggctgcatctgcaagggcgcatcggacaagtgcagctgctgtgcctgatgtgggggagagcctattcctgatgtaaatagagcgacgtgtacaaacctacagtttgtggggggttttttggtgctttttgttttgggtccaactctgacccgtttgctactacattcctagttattttccctatgaaataatacgtgaattataataaaagctgtcgactttaaaaaaaaaaaaaaaaa

SEQ ID NO: 5 - protein, 61 aa, bos taurus
MDPNCSCTAGESCTCAGSCKCKDCKCASCKKSCCSCCPVGCAKCAQGCVCKGASDKCSCCA

SEQ ID NO: 6 - DNA, 421 bp, bos taurus
gctccagcacgccccttcgacaccctcgccatcctttgctcagcagtctccggaccccagcctccagttcagctcgccatggatcccaactgctcctgcaccgcgggtgaatcctgcacgtgtgccggctcctgcaaatgcaaagattgcaagtgcgcctcctgcaagaagagctgctgctcctgctgccccgtgggctgtgccaagtgtgcccagggctgcgtctgcaaaggggcttcggacaagtgcagctgctgtgcctgaaggcggggagagcctgctccccggtgtaaatagaacaacgtgtacaaacctgcatattttttttttaatacaacctgacccgtttgttacatccctgttttttttttttttttttgtcctataaaatacaagaatgataataaaactggttggcttt

SEQ ID NO: 7 - protein, 61 aa, oryctolagus cuniculus
MDPNCSCPTGGSCSCAGSCTCKACRCPSCKKSCCSCCPVGCAKCAQGCVCKGASDKCSCCA

SEQ ID NO: 8 - DNA, 186 bp, oryctolagus cuniculus
atggatcccaactgctcctgccccactggcggctcctgcagctgtgctggctcctgcacctgcaaggcctgcagatgtccctcctgcaagaagagctgctgctcctgctgccctgtgggctgtgccaagtgtgcccagggctgtgtctgcaaaggggcctcggacaagtgcagctgctgcgcctga

SEQ ID NO: 9 - protein, 61 aa, mus musculus
MDPNCSCASDGSCSCAGACKCKQCKCTSCKKSCCSCCPVGCAKCSQGCICKEASDKCSCCA

SEQ ID NO: 10 - DNA, 556 bp, mus musculus
ggtcgtgcgcaggcccaggggcgtgtgctggccatatcccttgagccagaaaaagggcgtgtgcaggcggcgggggcgcgtgcatggtgccttccacccgggcggagcttttgcgctcgacccaatactctccgctataaaggtcgcgctccgcgtgcttctctccatcacgctcctagaactcttcaaaccgatctctcgtcgatcttcaaccgccgcctccactcgccatggaccccaactgctcctgtgcctccgatggatcctgctcctgcgctggcgcctgcaaatgcaaacaatgcaaatgtacttcctgcaagaaaagctgctgctcctgctgccccgtgggctgtgcgaagtgctcccagggctgcatctgcaaagaggcttccgacaagtgcagctgctgtgcctgaaggggggcggaggggtccccacatctgtgtaaatagaccatgtagaagcctagccttttttgtacaaccctgactcgttctccacaactttttctataaagcatgtaactgacaataaaagccgttgacttgattaattc

SEQ ID NO: 11 - protein, 61 aa, macaca fascicularis
MDPNCSCVAGDSCTCAGSCKCKECKCTSCKKSCCSCCPVGCAKCAQGCICKGASDKCNCCA

SEQ ID NO: 12 - DNA, 350 bp, macaca fascicularis
cccgactccagccgcctcttcaactcgccatggatcccaactgctcttgcgtcgccggtgactcctgcacctgcgccggctcctgcaagtgcaaagagtgcaaatgcacctcctgcaagaaaagctgctgctcctgctgccctgtgggctgtgccaagtgtgcccagggctgcatctgcaaaggggcgtcggacaagtgcaactgctgcgcctgatgctgggacagccctgctcccagatgtaaataatgcgacctctacaaacctggatttttttatgtacaaccctgatcgtttgctgcattcctttttctatgaaataatatgaatgataataaaacagctttgacg